SEQUENCE LISTING (8 sequences)

SEQ ID NO: 1 - DNA, 453 bp, Bacillus anthracis
  CDS (1)..(453)
atgacagaagattctttgcatctagataatcaactttgtttttctatttatgcttgttctagagaggttacacgcttttaccgaccgtatttagaagaaatgggcattacgtatccacagtacattactttactcgtactatgggagcaagatgggctaacagtaaaagaaattggagaacgtctatttttagattccggtacgttaacacctatgttaaaacgaatggaatcattaaaccttgtaaaacgcgttcgttccaaagaagatgagcgaaaagtgtgtattgaattaacagaacaaggaaaagatttacaagataaggcttgttcattgccgacaacgatggcaacaaatttaggaattacagaacaagaatatcgttctttattaattcaattaaataaactaattgaaacaatgaaaacaattaatgatagaaaaggggaataa

SEQ ID NO: 2 - protein, 150 aa, Bacillus anthracis
MTEDSLHLDNQLCFSIYACSREVTRFYRPYLEEMGITYPQYITLLVLWEQDGLTVKEIGERLFLDSGTLTPMLKRMESLNLVKRVRSKEDERKVCIELTEQGKDLQDKACSLPTTMATNLGITEQEYRSLLIQLNKLIETMKTINDRKGE

SEQ ID NO: 3 - protein, 145 aa, Bacillus anthracis
DQHNLKEQLCFSLYNAQRQVNRYYSNKVFKKYNLTYPQFLVLTILWDESPVNVKKVVTELALDTGTVSPLLKRMEQVDLIKRERSEVDQREVFIHLTDKSETIRPELSNASDKVASASSLSQDEVKELNRLLGKVIHAFDETKEK

SEQ ID NO: 4 - protein, 144 aa, Bacillus anthracis
DSLHLDNQLCFSIYACSREVTRFYRPYLEEMGITYPQYITLLVLWEQDGLTVKEIGERLFLDSGTLTPMLKRMESLNLVKRVRSKEDERKVCIELTEQGKDLQDKACSLPTTMATNLGITEQEYRSLLIQLNKLIETMKTINDR

SEQ ID NO: 5 - protein, 147 aa, Bacillus anthracis
MSDQHNLKEQLCFSLYNAQRQVNRYYSNKVFKKYNLTYPQFLVLTILWDESPVNVKKVVTELALDTGTVSPLLKRMEQVDLIKRERSEVDQREVFIHLTDKSETIRPELSNASDKVASASSLSQDEVKELNRLLGKVIHAFDETKEK

SEQ ID NO: 6 - protein, 132 aa, Bacillus anthracis
DQHNLKEQLCFSLYNAQRQVNRYYSNKVFKKYNLTYPQFLVLTILWDESPVNVKKVVTELALDTGTVSPLLKRMEQVDLIKRERSEVDQREVFIHLTDKSETIRPELSNASDKVASASSLSQDEVKELNRLL

SEQ ID NO: 7 - protein, 131 aa, Bacillus anthracis
DHMKLENQLCFLLYASSREMTKQYKPLLDKLNITYPQYLALLLLWEHETLTVKKMGEQLYLDSGTLTPMLKRMEQQGLITRKRSEEDERSVLISLTEDGALLKEKAVDIPGTILGLSKQSGEDLKQLKSAL

SEQ ID NO: 8 - protein, 148 aa, Mycobacterium tuberculosis
MGKGAAFDECACYTTRRAARQLGQAYDRALRPSGLTNTQFSTLAVISLSEGSAGIDLTMSELAARIGVERTTLTRNLEVMRRDGLVRVMAGADARCKRIELTAKGRAALQKAVPLWRGVQAEVTASVGDWPRVRRDIANLGQAAEACR

(19) United States
(12) Patent Application Publication
(10) Pub. No.: US 2007/0248581 A1
(43) Pub. Date: Oct. 25, 2007

(54) MGRA IS A REDOX REGULATOR OF ANTIBIOTIC SENSITIVITY AND VIRULENCE

(75) Inventors: Peng Chen, Chicago, IL (US); Chuan He, Chicago, IL (US)

Correspondence Address: FULBRIGHT & JAWORSKI L.L.P., 600 CONGRESS AVE., SUITE 2400, AUSTIN, TX, 78701, US

(73) Assignee: THE UNIVERSITY OF CHICAGO

(21) Appl. No.: 11/668,959

(22) Filed: Jan. 30, 2007

Related U.S. Application Data
(60) Provisional application No. 60/763,667, filed on Jan. 30, 2006.
(60) Provisional application No. 60/865,595, filed on Nov. 13, 2006.

(51) Int. Cl. C12N 1/36; A61K 35/74; C12Q 1/18; C12Q 1/68

(52) U.S. Cl. 424/93.42; 424/93.4; 435/245; 435/32; 435/6

(57) ABSTRACT

The present invention relates to the identification of a specific cysteine (Cys) residue in MgrA of Staphylococcus aureus and homologs in other bacteria that acts as a redox switch to regulate virulence of S. aureus and expression of bacterial genes that confer resistance to antibiotics. In addition, MgrA is found to be a key virulence determinant for S. aureus. Oxidative regulation of other Cys containing proteins in various bacteria has been observed, leading to the ability to modulate virulence and resistance of these organisms.

This application is related to U.S. Provisional Application Ser. Nos. 60/763,667 and 60/865,595 filed Jan. 30, 2006 and Nov. 13, 2006, respectively, the entire contents of which are hereby incorporated by reference.

### BACKGROUND OF THE INVENTION

1. Field of the Invention

The present invention relates to the fields of pathology and microbiology. More particularly, the present invention involves the identification of a redox sensing mechanism involving MgrA and MgrA homologs found in a variety of bacteria that regulate antibiotic sensitivity and virulence in bacteria.

2. Description of Related Art

Staphylococcal species are among the most robust of human pathogens and have a propensity for developing bacterial resistance. In less than two decades following the introduction of penicillin, methicilin and vancomycin, Staphylococcus species had arisen that were resistant to each of these drugs. Thus, given the widespread nature of this bacterium, it is clear that the mechanisms of overcoming bacterial drug resistance are critical to continued success in treatment.

MgrA is a transcription factor that regulates the expression of a number of protein efflux pumps involved in antibiotic resistance and formation of biofilms in Staphylococcus aureus (S. aureus). MgrA was first identified as regulating expression of type 8 capsular polysaccharide (CP8), nuclease, alpha-toxin, coagulase, protease, and protein A (Luong et al., 2003), a multidrug efflux pump NorA (Truong-Bolduc et al., 2003; Kaatz et al., 2005), and autolytic genes (Ingavale et al., 2003).

A recent transcription profiling study suggests that MgrA regulates 350 genes, many involved in virulence and metabolic regulation (Luong et al., 2006). Given that MgrA appears to play some role in drug resistance, it provides an interesting target for additional studies directed at elucidating its specific function, and is possibly a point of therapeutic intervention.

### SUMMARY OF THE INVENTION

Thus, in accordance with the present invention, there is provided a method of identifying a modulator of bacterial MgrA function comprising (a) providing an MgrA polypeptide or fragment thereof that (i) binds DNA and (ii) comprises a cysteine residue corresponding to that found at Cys12 of Staphylococcus aureus MgrA; (b) contacting said MgrA polypeptide or fragment with a candidate substance; and (c) assessing the binding of said MgrA polypeptide or fragment to a target DNA, wherein a change in the binding of said MgrA polypeptide or fragment to said target DNA, as compared to binding in the absence of said candidate substance, identifies said candidate substance as a modulator of bacterial MgrA function.

The MgrA may be from a Staphylococcus species, such as S. aureus or S. epidermidis, or from a Bacilles species, such as B. anthracis or B. cereus, or from a Mycobacterium species, such as M. tuberculosis. Further choices for the MgrA may be one from Streptococcus pneumoniae, Streptococcus pyogenes, Streptococcus agalactiae, Streptococcus viridans, Enterococcus faecalis, Enterococcus faecium, Clostridium botulinum, Clostridium perfringens, Clostridium tetani, Clostridium difficile, Listeria monocytogenes, Legionella pneumophila, Francisella tularensis, Pasteurella multocida, Brucella abortis biovar, Brucella suis, Brucella melitensis, Bordetella pertussis, Salmonella sp., Shigella sp., Eschericia coli, Vibrio sp. (V. alginolyticus), Klebsiella sp., Aeromonas sp., Plesiomonas sp., Rickettsiae sp., Chlamydiae sp., Ehrlichia sp., Mycoplasma sp., Helicobacter sp., Campylobacter sp., or Haemophilus sp. The candidate substance may be a peptide or a peptidomimetic, DNA, siRNA, antibody or antibody fragment, an inorganic metal salt or an organopharmaceutical. The MgrA polypeptide or fragment binding to DNA may be measured by, among other methods, a gel mobility shift assay, a South-Western blot, fluorescence anisotropy (FA), or FRET assay.

The method may be performed wherein at least steps (a) and (b) are performed in a cell free system, or wherein at least steps (a) and (b) are performed in a bacterial cell. The MgrA polypeptide or fragment may contain an oxidized Cys12 residue, or may contain a reduced Cys12 residue. The method may further comprise contacting said MgrA polypeptide or fragment with an oxidizing agent. In such cases, the oxidizing agent is added (i) prior to step (b) or (ii) after step (b) and before step (c). The oxidizing agent may be hydrogen peroxide, an organic hydroperoxide (e.g., cumene hydroperoxide), nitric oxide, dioxygen or superoxide.

Based on homology present between MgrA and other Cys-containing polypeptides, the inventors envision applying the preceding assay to other bacterial proteins such as MgrH1 and SarA, as well as other structurally-related bacterial proteins.

In another embodiment, there is provided a method of identifying a modulator of bacterial MgrA function comprising (a) providing an MgrA polypeptide or fragment thereof that (i) binds DNA and (ii) comprises a cysteine residue corresponding to that found at Cys12 of Staphylococcus aureus MgrA; (b) contacting said MgrA polypeptide or fragment with a target DNA; (c) contacting said MgrA polypeptide or fragment/DNA complex with a candidate substance; and (d) assessing the release of said MgrA polypeptide or fragment from said target DNA, wherein a change in the release of said MgrA polypeptide or fragment from said target DNA, as compared to release in the absence of said candidate substance, identifies said candidate substance as a modulator of bacterial MgrA function.

The MgrA may be as set forth above. The candidate substance may be a peptide or a peptidomimetic, DNA, siRNA, antibody or antibody fragment, an inorganic metal salt or an organopharmaceutical. The MgrA polypeptide or fragment binding to DNA may be measured by, among other methods, a gel mobility shift assay, a South-Western blot, fluorescence anisotropy, or FRET assay.

The method may be performed wherein at least steps (a) and (b) are performed in a cell free system, or wherein at least steps (a) and (b) are performed in a bacterial cell. The MgrA polypeptide or fragment may contain an oxidized Cys12 residue, or may contain a reduced Cys12 residue. The method may further comprise contacting said MgrA polypeptide or fragment with an oxidizing agent. In such cases, the oxidizing agent is added (i) prior to step (b) or (ii) after step (b) and before step (c). The oxidizing agent may be hydrogen peroxide, an organic hydroperoxide (e.g., cumene hydroperoxide), an organic hydroperoxide, nitric oxide, dioxygen or superoxide.

Based on homology present between MgrA and other Cys-containing polypeptides, the inventors envision applying the preceding assay to other bacterial proteins such as MgrH1 and SarA, as well as other structurally-related bacterial proteins.

In yet another embodiment, there is provided a method of identifying a modulator of bacterial MgrA function comprising (a) providing an MgrA polypeptide or fragment thereof that comprises Cys12; (b) contacting said MgrA polypeptide or fragment with a candidate substance; and (c) assessing the oxidation state of a cysteine residue corresponding to that found at Cys12 of Staphylococcus aureus in said MgrA polypeptide or fragment thereof, wherein a change in the oxidation state of Cys12 of said MgrA polypeptide or fragment, as compared to the oxidation state of Cys12 of said MgrA polypeptide or fragment in the absence of said candidate substance, identifies said candidate substance as a modulator of bacterial MgrA function.

The MgrA may be as set forth above. The candidate substance may be a peptide or a peptidomimetic, DNA, siRNA, antibody or antibody fragment, an inorganic metal salt or an organopharmaceutical. Assessing the oxidation state of Cys12 may comprise an assay using thiol reactive probes, such as 4-chloro-7-nitrobenz-2-oxa-1,3-diazole (NBD chloride; 4-chloro-7-nitrobenzofurazan) or 5,5′-dithiobis-(2-nitrobenzoic acid) (DTNB; Ellman's reagent).

The method may be performed wherein at least steps (a) and (b) are performed in a cell free system, or wherein at least steps (a) and (b) are performed in a bacterial cell. The MgrA polypeptide or fragment may contain an oxidized Cys12 residue, or may contain a reduced Cys12 residue. The method may further comprise contacting said MgrA polypeptide or fragment with an oxidizing agent. In such cases, the oxidizing agent is added (i) prior to step (b) or (ii) after step (b) and before step (c). The oxidizing agent may be hydrogen peroxide, an organic hydroperoxide (such as cumene hydroperoxide), nitric oxide, dioxygen or superoxide.

Based on homology present between MgrA and other Cys-containing polypeptides, the inventors envision applying the preceding assay to other bacterial proteins such as MgrH1 and SarA, as well as other structurally-related bacterial proteins.

In still another embodiment, there is provided an isolated and purified complex of bacterial MgrA and anhydrous tetracycline. The complex may be crystallized. The complex may further comprise DNA. In still a further embodiment, there is provided an isolated and purified complex of bacterial MgrA and DNA.

In yet another embodiment, there is provided a method of improving the efficacy of an antibiotic comprising contacting a bacterium with a drug that (i) increases MgrA or cysteine-containing MgrA homolog binding to DNA or (ii) inhibits MgrA or cysteine-containing MgrA homolog dissociation from DNA. The bacterium may be a Staphylococcus species, such as S. aureus and S. epidermidis, or a Bacilles species, such as B. anthracis or B. cereus, or a Mycobacterium species, such as M. tuberculosis. The bacterium may be located in an animal host, such as a human or a cow. The bacterium may be a multi-drug resistant strain. The MgrA homolog may be MgrH1 or SarA, as well as other structurally-related bacterial proteins.

In an additional embodiment, there is provided a method of improving the efficacy of an antibiotic comprising contacting a bacterium with a drug that reduces or inhibits oxidation of a cysteine residue corresponding to that found at Cys12 of Staphylococcus aureus MgrA. The bacterium may a Staphylococcus species, such as S. aureus or S. epidermidis, or from a Bacilles species, such as B. anthracis or B. cereus, or from a Mycobacterium species, such as M. tuberculosis. Further choices for the MgrA may be one from Streptococcus pneumoniae, Streptococcus pyogenes, Streptococcus agalactiae, Streptococcus viridans, Enterococcus faecalis, Enterococcus faecium, Clostridium botulinum, Clostridium perfringens, Clostridium tetani, Clostridium difficile, Listeria monocytogenes, Legionella pneumophila, Francisella tularensis, Pasteurella multocida, Brucella abortis biovar, Brucella suis, Brucella melitensis, Bordetella pertussis, Salmonella sp., Shigella sp., Eschericia coli, Vibrio sp. (V. alginolyticus), Klebsiella sp., Aeromonas sp., Plesiomonas sp., Rickettsiae sp., Chlamydiae sp., Ehrlichia sp., Mycoplasma sp., Helicobacter sp., Campylobacter sp., or Haemophilus sp.

In yet an additional embodiment, there is provided a method of treating a bacterial infection comprising administering to a subject an antibiotic and a drug that increases MgrA or cysteine-containing MgrA homolog binding to DNA or inhibits MgrA or cysteine-containing MgrA homolog dissociation from DNA. The bacterium may be as set forth above. The bacterium may be located in an animal host, such as a human or a cow. The bacterium may be a multi-drug resistant strain. The MgrA homolog may be MgrH1 or SarA, as well as other structurally-related bacterial proteins. The subject may be immunocomprised.

Another embodiment is a method of treating a bacterial infection comprising administering to a subject an antibiotic and a drug that reduces or inhibits oxidation of a cysteine residue corresponding to that found at Cys12 of Staphylococcus aureus MgrA. The bacterium may be as set forth above. The subject may be immunocomprised. It also is contemplated that this embodiment could be further improved by the addition of one or more antivirulence compounds such as RNAIII inhibiting peptides (RIPs). Such molecules are described in U.S. Pat. No. 6,291,431, incorporated herein by reference.

Yet another embodiment includes a method of reducing the virulence of a bacterium comprising contacting said bacterium with an agent that inhibits the expression and/or function of MgrA or a cysteine-containing MgrA homolog. The bacterium may be as set forth above. The inhibitor may be an organopharmaceutical, protein, peptidomimetic, peptide, an inorganic metal salt, or nucleic acid. The bacterium may be located in an animal host, such as a human or a cow. The bacterium may be a multi-drug resistant strain. The MgrA homolog may be MgrH1, or SarA, as well as other structurally-related bacterial proteins.

Still a further embodiment is a method of reducing the virulence of a bacterium comprising contacting said bacterium with an agent that promotes oxidation of a cysteine residue corresponding to that found at Cys12 of Staphylococcus aureus MgrA. The bacterium may be as set forth above. The inhibitor may be an organopharmaceutical, protein, peptide, peptidomimetic, an inorganic metal salt or nucleic acid. The bacterium may be located in an animal host, such as a human. The agent may induce a conformational change that would disrupt the Cys12 interaction with its binding partners, modify Cys12 (e.g., alkylate) or bind near Cys12, thereby disrupting H-bonding, causing dissociation of MgrA from DNA. The bacterium may be a multi-drug resistant strain.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Plate assay for resistance of the mgrA- and mgrH- strains to ciprofloxacin (0.20 μg/ml), norfloxacin (0.80 μg/ml), ethidium bromide (5.0 μg/ml), and chloramphenicol (1.0 μg/ml). The wild-type Newman strain was used as the control.

FIG. 2: Effect of the mgrA mutation on the virulence of S. aureus tested using the murine abscess model of infection. Each circle stands for one animal experiment. The horizontal bars indicate the mean (the Colony Forming Unit (CFU) number was converted to log CFU and the arithmetic mean was obtained) and the dashed line represents the limit of detection, which is 50 CFU/ml in this case (since 20 82 l of homogenates (1 ml total) were used for colony enumeration).

FIGS. 3A-C: Crystal structure of MgrA. (FIG. 3A) Ribbon representation of the MgrA dimer. Some basic residues on the DNA-binding domain are shown together with one ordered sulfate anion per monomer. The distance between two α4 helices is ˜34 Å, which correlates to the spacing between two consecutive sections of the major groove of a B-form duplex DNA. (FIG. 3B) Molecular modeling of MgrA-DNA complex. The two α4 helices fit right into the major groove of a B-form duplex DNA. The sulfate anions overlap with the DNA backbone. (FIG. 3C) Overlay of the dimerization domain of the MgrA and E. coli MarR structures.

FIGS. 4A-C: The unique Cys12 residue in MgrA. (FIG. 4A) The location of Cys12 in one monomer (Cys12′) is highlighted in the figure. (FIG. 4B) Close up of the reduced Cys12 site in MgrA. Potential hydrogen-bonds involving Cys12′ (from one monomer), Tyr26, Tyr38 and Ser113 (other monomer) are shown as dashed lines, with distances labeled. Additional hydrophobic side chains on α2 are also shown. (FIG. 4C) Sequence alignment of MgrA with B. subtilis OhrR. The conserved redox active Cys residues are boxed in dark grey, with other highly conserved residues in light grey. Tyr26, Tyr38 and Ser113 are denoted by asterisks.

FIGS. 5A-B: Oxidation of Cys12 in MgrA in vitro. (FIG. 5A) Cys-sulphenic acid formed in vitro from Cys12 oxidation was trapped by the NBD-Cl assay. Reaction of the oxidized MgrA with NBD-Cl (solid line): Cys-S(O)-NBD absorbs at 347 nm; reaction of the reduced MgrA with NBD-Cl (dashed line): Cys-S-NBD absorbs at 420 nm. (FIG. 5B) Quantification of free thiol in MgrA upon oxidation (open bars) and reduction (filled bars) by the DTNB assay. The reduced form of purified MgrA contains one free thiol per monomer. This form of protein was treated with 4 equivalents of CHP, H₂O₂ or KO₂ for 10 min or with air for over 3 h to generate the oxidized MgrA. A MgrAC12S mutant protein was also assayed as the control. Error bars indicate s.d.

FIG. 6: Electrophoretic mobility shift assay demonstrating the effect of oxidation on the DNA binding of MgrA. Purified MgrA (or MgrAC12S) protein was incubated with a 40 bp oligonucleotide (0.4 pmol per 20 μl reaction) containing the MgrA binding sequence on the sarV promoter region. Lane a1 (or b1) contained 0 μM MgrA (or MgrAC12S) and lanes a2-a9 (or b2-b9) contained 2 μM MgrA (or MgrAC12S). Either CHP (7.5 mM) or H₂O₂ (15 mM) was added to the binding assay which was incubated for 30 min at room temperature. When indicated, 50 mM DTT was then added into the solution and incubation continued at room temperature for 30 min before samples were applied for the shift assay.

FIGS. 7A-B-C: Monitoring in vivo effects of MgrA oxidation. The change of susceptibility levels of S. aureus strains toward ciprofloxacin and vancomycin under oxidative stress. The antibiotic resistance levels were tested in the absence (−H₂O₂) or presence (+H₂O₂) of 100 μM H₂O₂ by a plate sensitivity assay (FIG. 7A) and were also determined in the absence (−PQ) or presence (+PQ) of 25 μM paraquat by a 96-well-plate sensitivity assay (FIG. 7B). Under normal growth conditions (control) the five strains did not show noticeable differences. The wild-type strain and the mgrA mutant strain complemented with pYJ335-His-mgrA showed higher susceptibility towards ciprofloxacin and vancomycin. Under oxidation conditions, both strains exhibited increased resistance toward these antibiotics comparable to that of the mgrA mutant strain. In control experiments, the pYJ335-His-mgrAC12S-containing mutant stain did not change its susceptibility towards CIP and VCM under normal or oxidative conditions. (FIG. 7C) Induction of norA, a gene regulated by mgrA, by oxidative stress. β-Galactosidase activity of strains containing the norA-lacZ reporter fusion was determined in the wild-type (Newman) and mgrA- mutant strain and expressed in MUG units (MUG: 4-methylumbelliferyl-β-D-galactopyranoside; 1 MUG unit=1 pmol of MUG cleaved by β-galactopyranoside per min per OD600). Empty bars are non-treated cultures. Results are mean ±s.d. from three independent experiments performed in duplicate.

FIG. 8: MgrA uses an oxidation sensing mechanism to regulate gene activation in S. aureus.

FIG. 9: Sequence alignment of MgrA and SarA. Absolutely conserved residues are shaded (the unique Cys residues are more darkly shaded). Other conserved residues are marked with asterisks.

FIGS. 10A-B-C: SarA uses an oxidation sensing mechanism. (FIG. 10A) Treatment of SarA with CHP leads to dissociation of SarA from DNA, as monitored by fluorescence anisotropy (FA). (FIG. 10B) Control with SarA C9S mutant protein. (FIG. 10C) Monitoring in vivo effects of SarA oxidation by qRT-PCR. A virulence gene splB is known to be repressed by SarA. Treatment of the Newman strain with H₂O₂ (0.4 mM) for 30 min led to activation of this gene.

FIGS. 11A-B: Strategies for tuning MgrA with small molecules in S. aureus. (FIG. 11A) Alkylation of Cys12 causes disruption of the hydrogen-bonding network around this residue and dissociation of MgrA from DNA. This event will suppress the virulence of S. aureus. (FIG. 11B) It is also possible to have small molecules that bind MgrA and cause a similar conformation change, achieving the same goal.

FIG. 12: Electrophoretic mobility shift assay demonstrating the effect of Cys12 alkylation on the DNA binding of MgrA. Lanes 1 and 2, purified MgrA (2 μM) with a 40 bp oligonucleotide (0.4 pmol) containing the MgrA binding sequence on the sarV promoter region; lanes 3 and 4, MgrA was treated with 100 equivalents of phenyl vinyl sulfone (7) for 30 min (lane 3) or 60 min (lane 4) before mixed with DNA and applied for the shift assay. FIGS. 13A-B: The Cys-alkylator phenyl vinyl sulfone (PVS) has similar effects on MgrA as hydrogen peroxide. (FIG. 13A) Treatment of the Newman strain with PVS turned on resistance of the pathogen towards vancomycin. (FIG. 13B) The norA gene is repressed by MgrA; however, treatment of the Newman strain with either H₂O₂ or PVS can turn on this gene.

FIG. 14: Sequence alignment of MgrA, MgrH1 and E. coli MarR. Conserved residues between MgrA and MgrH1 are shaded (the unique Cys residue is more darkly shaded). Residues conserved in all three sequences are marked with asterisks.

FIG. 15: Small molecules to “trick” S. aureus into the low virulence form. Cause infections and diseases.

ACCESSION NOS. FOR MgrA OF VARIOUS BACTERIA*

| Organism | Whole Genome | Locus Tag | Protein |
|---|---|---|---|
| S. aureus | NC_002758 | SAV0686 | NP_371210 |
| B. anthracis | NC_007530.2 | Bant_01005352 | ZP_00394795 |
| B. cereus | AE016877 | BC_4474 | AAP11387 |
| M. tuberculosis | NC_000962 | Rv1049 | NP_215565 |
| P. aeruginosa | NZ_AAKW1000023.1 | | |
| S. agalactiae | NZ_AAJO01000039.1 | | |
| S. epidermidis | NC_002976.3 | | |
| S. epidermidis | NC_004461 | | |
| B. abortis | NC_006933.1 | | |
| C. perfringens | NC_003366.1 | | |
| E. faecium | AAAK03000030.1 | | |
| L. moncytogenes | NZ_AADQ01000010.1 | | |
| V. alginolyticus | NZ_AAPS01000001.1 | | |

*Each of the preceding accession nos. and their respective sequences are hereby incorporated by reference.

### DESCRIPTION OF ILLUSTRATIVE EMBODIMENTS

The present invention relates to the identification, in several species of bacteria, of a conserved cysteine (Cys) residue in MgrA, as well as homologs containing a similarly situated Cys residue. The cysteine residue serves as a redox sensor and controls the association of these proteins with target DNA, for example, to regulate the expression of efflux pumps. Disruption of the MgrA gene results in reduced sensitivity to antibiotics, but also causes reduced virulence in vivo. These and other aspects of the invention are described in greater detail below.

1. Bacteria

A. Staphylococcus

Within the family Micrococcaceae, the human pathogenic genus Staphylococcus can be separated from the nonpathogenic genus Micrococcus by various tests, including (1) anaerobic acid production from glucose, (2) sensitivity to 200 μg/ml lysostaphin or to 100 μg furarazolidone, and (3) production of acid from glycerol in the presence of 0.4 μg/ml erythromycin, all these tests being positive in the case of staphylococci. Further subclassification into the three main species is of considerable clinical importance (i.e., S. aureus, Staphylococcus epidermidis, and Staphylococcus saprophyticus).

Once the Staphylococcus has been differentiated as Staphylococcus aureus, it is necessary to determine if the S. aureus is methicillin resistant. Older methods such as resistance phenotype, bacteriophage typing, immunoserology, and serotyping of coagulase can be used to type S. aureus. More recently, these methods have been replaced by electrophoretic protein typing, multilocus enzyme electrophoresis, and various genetic techniques, including plasmid analysis, restriction endonuclease analysis of chromosomal DNA, restriction fragment length polymorphisms, ribotyping, nucleotide sequence analysis, and many others.

B. Sensitivity and Resistance Patterns of Staphylococcus aureus

Typically, antibiotic resistance in S. aureus may be mediated by chromosomes or plasmids. Staphylococci exchange genetic material by various mechanisms, including transduction and cell-to-cell contact (Udo et al., 1992). Recent evidence is accumulating in favor of transfer of plasmids between S. aureus and S. epidermidis.

i) β-Lactamase-Mediated Resistance

In this case, the microorganism produces an extracellular enzyme that inactivates the antibiotic by opening its β-lactam ring before it has caused irreversible changes in the bacterium itself. This mutual time-dependent interaction implies that the presence of a large number of microorganisms will outweigh the effect of the antibiotic and accelerate its destruction.

ii) Intrinsic Resistance

Intrinsic resistance, also called methicillin resistance, encompasses all β-lactams, including cephalosporins. Methicillin resistance is defined as an oxacillin minimum inhibitory concentration (MIC) of 4 mg/liter or greater or a methicillin MIC of 16 mg/liter or greater. Several methods are available to detect methicillin resistance, which is improved by supplementing the media with sodium chloride, a low temperature of 30° C., and a high inoculum. Determination in agar dilution is performed in Mueller-Hinton medium containing 5% sodium chloride and incubated at 37° C. for 18 hours or in Mueller-Hinton medium without sodium chloride at 30° C. In liquid media, the MIC is determined in Mueller-Hinton medium containing 2% sodium chloride with an inoculum of 5×10⁵ colony-forming units (cfu) per milliliter. When disk diffusion is used, an oxacillin disk charged with 1 μg is applied to Mueller-Hinton agar containing 5% sodium chloride and incubated at 37° C. or without sodium chloride and incubated at 30° C. with an inoculum of 10⁶ cfu/ml. These methods have been adapted for various automated devices and kits used in the clinical laboratory. Because of difficulty in interpretation with all the methods mentioned earlier, particularly in the presence of heteroresistance, many laboratories use a screening plate containing Mueller-Hinton agar with 6 mg/liter of oxacillin and 4% sodium chloride inoculated with 10⁴ cfu per spot and incubated at 37° C. for 24 hours.

Other characteristics of methicillin resistant S. aureus (MRSA) include penicillinase production and a full armamentarium of pathogenic factors, including coagulase and DNase. Also, catalase seems to be present in enhanced amounts (Peacock, 1981). Numerous in vitro studies have shown MRSA to be fully virulent (Kinsman et al., 1985) and to have an intraphagocytic survival potential (Vaudaux, 1979) and a lethality in animal studies (Peacock et al., 1981) similar to that of their sensitive congeners.

Staphylococci normally have at least two essential penicillin-binding proteins (PBPs) bound to the internal cytoplasmic membrane; these PBPs have enzymatic activities and are responsible for cross-linking of the peptidoglycan cell wall. Staphylococci can become resistant to all β-lactams, including β-lactamase inhibitor combinations, and to all cephalosporins and carbapenems by the acquisition of a chromosomal mecA gene, which encodes an alternative supplementary target called PBP 2a (or PBP 2) that has low affinity for β-lactams. This abnormal PBP 2a continues to function when PBP 1, 2, and 3 have been inactivated by β-lactam antibiotics and generates a stable peptidoglycan. Phenotypic expression of the mecA gene varies among staphylococci. In some strains, only a minority of cells express resistance, and they are therefore called heteroresistant; in other strains, expression is homogeneous. Expression of the mecA gene is regulated by various auxiliary factors, including five Fem factors (factor essential for resistance to methicillin) and others (Berger-Bachi, 1994). The mecA gene is located on a large 30- to 40-kilobase DNA element (mec) of unknown origin that contains many other genes and is virtually identical in all species of staphylococci. It is flanked by insertion sequence-like elements (IS431 and IS257) that appear to have been acquired by horizontal gene transfer and act as a trap for additional unrelated drug resistance genetic determinants, thereby leading to multiple resistance.

iii) Borderline Oxacillin-Resistant Staphylococcus aureus

Resistance to oxacillin is seen in strains harboring the mecA gene and in other strains without the mecA gene. The latter resistance is due to hyperproduction of β-lactamase, and such organisms are called borderline oxacillin-resistant S. aureus. This hyperproduction of β-lactamase requires high sodium chloride concentrations (Sierra-Madero et al., 1988) and does not induce a higher MIC in normal testing conditions.

iv) Other Antibiotics

S. aureus possesses a remarkable number of mechanisms for resisting antibacterial action. Thus, depending on the local epidemiologic conditions, 5 to 20% of isolates are resistant to the antibacterial agents commonly used in staphylococcal infections such as erythromycin, lincomycin, and clindamycin. This percentage seems to be lower for fusidic acid, although clinical experience with this drug is limited. Aminoglycoside-resistant strains have been described with increasing frequency. Rifampin, which is remarkably active against S. aureus, cannot be used as a single agent because of a high one-step mutation rate of 10⁻⁷ to 10⁻⁸ to resistance (Moorman, 1981).

Resistance to fluoroquinolones has been found in methicillin sensitive (Kaatz et al., 1991) and methicillin resistant strains (Murakami et al., 1989). Both altered gyrase and energy-dependent efflux mechanisms are implied (Kaatz et al., 1991).

v) Glycopeptide-Resistant Staphylococcus aureus

A new threat has emerged: S. aureus resistant to glycopeptides (e.g., vancomycin). Because their MICs have been shown to be between 2 and 8 μg/ml, they have thus far been named intermediately vancomycin-resistant strains. They usually show heterogeneous resistance to vancomycin on population analysis and have higher MICs to teicoplanin. As opposed to vancomycin-resistant enterococci, they do not possess vanA, vanB, or vanc resistance genes.

Biochemical analysis has shown intermediately vancomycin-resistant S. aureus to produce increased amounts of cell wall precursors, express increased amounts of the penicillin-binding protein PBP 2, and have increased transglycosylation activity (Maranan et al., 1997). S. aureus intermediately resistant to vancomycin can be obtained by a stepwise increase in vancomycin concentration. It is presently hypothesized that these S. aureus strains “soak up” the antibiotic in their thickened cell walls. They are usually methicillin resistant, but sensitive to the oxazolidinones and to quinupristin/dalfopristin.

C. Bacillus

Bacilles species are rod-shaped, endospore-forming aerobic or facultatively anaerobic, Gram-positive bacteria; in some species cultures may turn Gram-negative with age. The many species of the genus exhibit a wide range of physiologic abilities that allow them to live in every natural environment. Only one endospore is formed per cell. The spores are resistant to heat, cold, radiation, desiccation, and disinfectants. Bacillus anthracis needs oxygen to sporulate; this constraint has important consequences for epidemiology and control. In vivo, B. anthracis produces a polypeptide (polyglutamic acid) capsule that protects it from phagocytosis. The genera Bacilles and Clostridium constitute the family Bacillaceae. Species are identified by using morphologic and biochemical criteria.

The virulence factors of B. anthracis are its capsule and three-component toxin, both encoded on plasmids. B. cereus produces numerous enzymes and aggressins. The principal virulence factors are a necrotizing enterotoxin and a potent hemolysin (cereolysin). Emetic food poisoning probably results from the release of emetic factors from specific foods by bacterial enzymes.

D. Mycobacterim

Both leprosy and tuberculosis, caused by Mycobacterium leprae and Mycobacterium tuberculosis respectively, have plagued mankind for centuries. With the emergence of antibiotic resistant strains of tuberculosis, research into Mycobacteria has become all the more important in combating these modem mutants of ancient pathogens.

Both the genomes of Mycobacterium tuberculosis and Mycobacterium leprae have been sequenced with hopes of gaining further understanding of how to defeat the infamously successful pathogens. The genome of M. tuberculosis is 4,411,522 base pairs long with 3,924 predicted protein-coding sequences, and a relatively high G+C content of 65.6%. At 4.4 Mbp, M. tuberculosis is one of the largest known bacterial genomes, coming in just short of E. coli, and a distant third to Streptomyces coelicolor.

The genome of Mycobacterium leprae is 3,268,203 base pairs long, with only 1,604 predicted protein-coding regions, and a G+C content of about 57.8%. Only 49.5% of the M. leprae genome contains open reading frames (protein-coding regions), the rest of the genome is comprised of pseudogenes, which are inactive reading frames with recognizable and functional counterparts in M. tuberculosis (27%), and regions that do not appear to be coding at all, and may be gene remnants mutated beyond recognition (23.5%). Of the genome of M. tuberculosis, 90.8% of the genome contains protein-coding sequences with only 6 pseudogenes, compared to the 1,116 pseudogenes on the M. leprae genome.

E. Pseudomonas

The genus Pseudomonas is characterized by Gram-negative rods that utilize glucose oxidatively. Members are classified into five groups based on ribosomal RNA homology. These bacteria are resistant to most antibiotics and are capable of surviving in very harsh conditions tolerated by very few other organisms. They also are known to produce a coating that helps protect the bacterium from outside agents. Pseudomonas is often found in hospitals and clinics and, not surprisingly, is a major cause of nosocomal infections. It often targets immunocompromised individuals, such as burn victims and individuals on respirators or with indwelling catheters. Infection sites are varied and include the urinary tract, blood, lungs, and pharynx. However, because it is non-invasive, it tends not to be found in healthy individuals.

Pseudomonas aeruginosa is the most common member of its genus, distinguished from other species of Pseudomonas by its ability to grow at 42° C., produce bluish (pyocyanin) and greenish pigments, and exhibit a characteristic fruity odor. The pathogenicity involves several toxins and chemicals that the bacterium secretes upon infection. The presence of a lipopolysaccharide layer serves to protect the organism as well as aid in cell adherence to host tissues. Lipases and exotoxins secreted by the organism then procede to destroy host cell tissue, leading to complications often associated with infection. P. aeruginosa prefers moist environments, and will grow on almost any laboratory medium. Pseudomonas infections are usually treated with a combination of antibiotics, e.g., an anti-pseudomonal penicillin and an aminoglycoside.

F. Other Bacteria

In addition to the bacteria discussed above, the inventors disclose methods for drug screening, methods for increasing bacterial sensitivity to antibiotics, and methods of reducing bacterial virulence for a variety of other bacteria. Such bacteria include Streptococcus pneumoniae, Streptococcus pyogenes, Streptococcus agalactiae, Streptococcus viridans, Enterococcus faecalis, Enterococcus faecium, Clostridium botulinum, Clostridium perfringens, Clostridium tetani, Clostridium difficile, Listeria monocytogenes, Legionella pneumophila, Francisella tularensis, Pasteurella multocida, Brucella abortive, Brucella suis, Brucella melitensis, Bordetella pertussis, Salmonella sp., Shigella sp., Eschericia coli, Vibrio sp., Klebsiella sp., Aeromonas sp., Plesiomonas sp., Rickettsiae sp., Chlamydiae sp., Ehrlichia sp., Mycoplasma sp., Helicobacter sp., Campylobacter sp., and Haemophilus sp.

2. Bacterial Targets Affected by Oxidation

As discussed above, the inventors initially observed that a S. aureus protein designated MgrA contained a single cysteine residue which played an important role in the regulation of virulence and antibiotic resistance. In assessing homologies with other bacterial proteins, the inventors have identified other candidates for virulence/resistance factors that also appear to be regulated by oxidation of Cys residues.

A. MgrA

MgrA is a transcription factor that regulates an large number of bacterial genes, including those involved in virulence and antibiotic resistance. For example, it regulates a number of protein efflux pumps involved in antibiotic resistance and formation of biofilms in S. aureus. The MgrA protein was discovered only recently (2003) by three independent groups. Hooper's group at Harvard Medical School was studying the NorA multidrug resistance efflux pump that causes resistance to the fluoroquinolone antibiotics. They noticed binding of a ˜17.6 kDa protein to the promoter of the norA gene. Subsequently, they showed that activation of norA is regulated by this protein, named NorR (MgrA) (Truong-Boldac et al., 2003). Cheung's group at Dartmouth Medical School was working on identifying factors that regulate virulence and autolysis in S. aureus. During their screen of transposon mutants, they discovered that knocking out a gene they named rat (mgrA) led to a substantial suppression of known autolytic regulators. Gel shift experiments confirmed that the Rat (MgrA) protein binds to the lytRS and arlRA promoters and serves as a negative regulator of autolysin genes in S. aureus (Ingavale et al., 2003). Lee's group at University of Kansas Medical Center also identified MgrA as a key regulator of type 8 capsular polysaccaride virulence factors through a similar screen of transposon mutants (Luong et al., 2003). The MgrA protein has a region of significant homology, including a helix-turn-helix DNA-binding motif, with Escherichia coli MarR family transcriptional regulators. In addition, SDS-PAGE analyses showed that the mgr locus affected extracellular protein production, suggesting a global regulation of protein synthesis (Luong et al., 2003).

NorA is a Staphylococcus aureus multidrug transporter that confers resistance to structurally distinct compounds. The MgrA global regulatory protein is reported to augment NorA expression when MgrA is overexpressed from an undefined plasmid-based promoter (Kaatz et al., 2005). In an analysis of the resistance mechanisms of an mgrA mutant, two genes encoding previously undescribed transporters, NorB and Tet38 were identified. Both norB and tet38 transcription was negatively regulated by MgrA (Truong-Bolduc et al., 2005). Significantly, a recent transcription profiling study suggests that MgrA regulates ˜350 genes, many of which are involved in virulence regulation and metabolic regulation (Luong et al., 2006).

B. MgrH1

MgrH1 was first recognized in the S. aureus genome as a remote homologue of the virulence regulator SarA and was named SarZ (Cheung et al., 2004). The function of this protein has never been characterized. MgrH1 was identified, along with MgrA, in a search for sequence homologs of E. coli MarR in the S. aureus genome. MgrH1 does not regulate antibiotic resistance but affects virulence, and it shares the same sensing mechanism as MgrA. A transcription profiling experiment was performed and MgrH1 was shown to regulate ˜80 proteins involved in virulence and defense functions. A key set of proteins regulated by MgrH1 are those involved in anaerobic energy production.

C. SarA

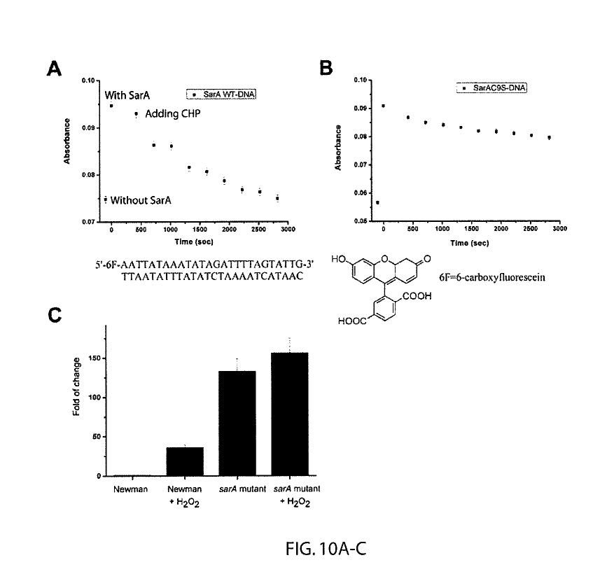

Among all the MarR type regulators in S. aureus, the unique Cys residue is found in three proteins, MgrA, MgrH1, and SarA. While the inventors have shown that MgrA and MgrH1 are highly homologous in sequence and quite susceptible to oxidation, SarA is less homologous to MgrA, but still possesses a lone Cys residue, Cys9 (FIGS. 10A-C). A previous random mutagenesis study has identified this Cys residue to be important in SarA-based regulation (Liu et al., 2006). Expression of a SarAC9A mutant protein in the sara⁻mutant strain led to activation of sarA, spa, and aur, all regulated by SarA. No activation was observed in a control experiment with the wild type SarA. Despite this finding, the exact role of Cys9 in SarA remains unclear. Using fluorescence anisotropy (FA) experiments with a fluorescein-labeled DNA known to be recognized by SarA, the inventors showed that, upon binding to SarA, the complex gave a higher FA readout, and that upon treating with CHP, SarA dissociated from DNA. In a control experiment with the C9S mutant SarA, CHP did not lead to a significant change of FA, confirming the oxidation dissociation mechanism of SarA.

The inventors also monitored activation of a virulence gene splB, known to be repressed by SarA, in S. aureus. As shown in qRT-PCR experiments, activation of this gene was repressed in the Newman strain, and the sarA mutant strain exhibited a higher activation level of this gene. When the Newman strain was treated with 0.4 mM H₂O₂ for 30 min, the splB gene was activated by oxidation, confirming the in vivo oxidation sensing proposed for SarA. See FIGS. 10A-C and Examples.

D. Other MgrA Homologs

The following bacterial proteins, classified as MgrA homologs, are proposed targets for cysteine-based oxidation modulation. The S. epidermidis homolog is shown in SEQ ID NO:22. SEQ ID NOS:9 and 10 and SEQ ID NOS: 11/12 are Pseudomonas aeruginosa proteins of unknown function. Both proteins contain a lone cysteine motif (LCF). Two Streptococcus agalactiae proteins of unknown function also exhibit this motif (SEQ ID NO:13 and 14). Clostridium perfringens exhibits an LCF-containing gene (SEQ ID NO:15). Enterococcus faecium contains a LCF motif protein (SEQ ID NO:16), as does Vibrio alginolyticus. Listeria monocytogenes, Brucella abortis biovar and Bacilles anthracis contain other LCF motif proteins (SEQ ID NO:20 and 21, SEQ ID NO:17 and 18 and SEQ ID NO:19 respectively).

3. Screening Assays

The present invention contemplates the screening of compounds for the ability to inhibit MgrA or MgrA homolog expression or function, to induce MgrA or MgrA homolog dissociation from DNA, to prevent or reduce dissociation of MgrA or an MgrA homolog from DNA, to activate oxidation or perform other irreversible modifications, such as alkylation, of a cysteine residue in the N-terminal portion of MgrA or an MgrA homolog, or to promote oxidation of or prevent reduction of an oxidized cysteine residue in the N-terminal portion of MgrA or an MgrA homolog. Alternatively, reversable inhibition may be achieved by nitrosylation of a cysteine. In the screening assays of the present invention, several different types of compounds will be screened for these basic biochemical activities, and then may be further tested for their ability to affect gene expression of MgrA- or MgrA homolog-regulated proteins. Yet further, compounds may be tested for their ability to increase bacterial sensitivity to antibiotics and/or to reduce bacterial virulence by tuning the function of MgrA.

A. Assay Formats

The present invention contemplates the use of both isolated MgrA or MgrA homolog polypeptides and fragments thereof, as well as in cyto assays using whole bacteria. Antimicrobial activity may include bacteriostatic (e.g., inhibition of growth) and/or bactericidal (e.g., death of bacteria) activities, as well as reductions in virulence. These assays may make use of the following format and may vary slightly depending upon the candidate substance.

For example, one such embodiment of the present invention is directed to a method of screening for agents comprising:
(a) providing an MgrA polypeptide, homologue or fragment thereof;
(b) contacting the MgrA, homologue or fragment with a candidate drug and DNA, either separately or simultaneously; and
(c) assessing affects on DNA binding by MgrA, homologue or fragment,
   wherein a change in the binding, as compared to that seen in the absence of the candidate drug, indicates that the candidate drug is an antimicrobial agent. The MgrA or MgrA homolog may be oxidized or alkylated at a cysteine positioned near the N-terminus of the molecule, or may be contacted with an oxidizing agent prior to or following contact with the candidate drug. This entire assay may be performed on a whole bacterium rather than an isolated MgrA or MgrA homolog molecule.

A variety of read-outs may be utilized to assess DNA binding by MgrA or MgrA homolog. Gel mobility shift assays can be used to determine binding of proteins to DNA due to the decrease in electrophoretic mobility of DNA when bound by proteins. Another assay to measure interaction of molecules involves FRET, or fluorescence resonance energy transfer. FRET assays may utilize labeling of the DNA (or MgrA or MgrA homolog) with a first molecule that can be quenched by a second reagent that is linked to the MgrA or MgrA homolog (or DNA, where the MgrA is labeled with the first molecule). When the two agents are adjacent, as in the case of DNA binding by MgrA or MgrA homolog, the signal from the first molecule is “quenched” and no signal is observed. Upon dissociation of MgrA or MgrA homolog from DNA, however, the quenching effect is lost and signal is observed.

The inventor has developed an assay to use fluorescence anisotropy to monitor dissociation of SarA from DNA. Another more “high throughput” method under development uses an environmentally-sensitive fluorophore called aminophenoxazone (AP). The excitation and emission wavelengths of this fluorophore are extremely sensitive to polarity of the environment (Coshen et al., PNAS, 2005, 102, 965). The fluorophore can be incorporated into the middle of the MgrA binding DNA sequence using a convertible nucleoside method. Binding of MgrA to DNA will change the environment of AP and shift its emission wavelength. The optimum position of AP on DNA can be obtained by the docking structure of MgrA on DNA. This position will also be systematically varied in synthetic DNA to achieve the optimum response. Then, small molecule-mediated dissociation of MgrA from DNA can be readily monitored by fluorescence change.

In a distinct embodiment, one may simply assess the oxidation state of the aforementioned cysteine residue by:
(a) providing an MgrA polypeptide, homologue or fragment thereof, wherein said MgrA, homologue or fragment comprises a cysteine positioned near the N-terminus of the molecule;
(b) contacting the MgrA polypeptide, homologue or fragment with a candidate drug and an oxidizing agent, either separately or simultaneously; and
(c) assessing affects on oxidation of the cysteine in MgrA, homologue or fragment,
   wherein a change in the oxidation state, as compared to that seen in the absence of the candidate drug, indicates that the candidate drug is an antimicrobial agent. This entire assay may also be performed on a whole bacterium rather than an isolated MgrA, homologue or fragment molecule.

Common assays to assess oxidation state include DTNB (5,5′-Dithiobis(2-nitrobenzoic acid); Ellman's Reagent) and NBD (nitrobenzofuran) assays (Donato et al,. 2004); Liu et al., 2004; Chougnet et al., 2003; Dunn et al., 2002; Nakamura et al., 2001; Thapliyal and Maru, 2001; Roser and Thomas, 2000; Kobayashi et al., 1998). The target Cys residue could also be alkylated which may disrupt its hydrogen-bonding interaction with its partners. This could lead to MgrA dissociation from DNA. In addition, a small molecule could non-covalently bind to MgrA and also induce its dissociation from DNA.

B. Candidate Substances

One may acquire, from various commercial sources, small molecule libraries that are believed to meet the basic criteria for useful drugs in an effort to use “brute force” to identify useful compounds. Screening of such libraries, including combinatorially generated libraries, is a rapid and efficient way to screen large number of related (and unrelated) compounds for activity. Combinatorial approaches also lend themselves to rapid evolution of potential drugs by the creation of second, third and fourth generation compounds modeled on active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, peptidomimetic, nucleic acid molecule, small molecule or any other compounds that may be designed through rational drug design starting from known antimicrobial compounds.

Other suitable candidate substances include peptides, antisense molecules, siRNAs, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

C. Determination of Antimicrobial Susceptibility

In the present invention, candidate drugs or substances may be initially screened in cell free systems, but may then be screened for their ability to alter virulence or drug sensitivity of a microbe per se, e.g., bacterial cells. A number of in vitro methods for determining antimicrobial susceptibility are available. The E test, for example, uses diffusion of a continuous concentration gradient of an antimicrobial agent (or two agents) from a plastic strip into an agar medium to yield quantitative measurements of antimicrobial susceptibility.

Other quantitative data are also provided by methods that incorporate serial dilutions of antimicrobials in agar-containing or broth culture media. The lowest concentration of the antimicrobial agent that prevents visible growth after an 18- to 24-hour incubation period is the minimal inhibitory concentration (MIC). The minimal bacterial concentration (MBC) or minimal lethal concentration (MLC) may be determined in broth dilution tests by subculturing the containers that show no growth onto antibiotic-free agar-containing media. The lowest concentration of antimicrobial that totally suppresses growth on antibiotic-free media (or results in a 99.9% or greater decline in colony count) after overnight incubation is known as the MBC (or MLC). The aforementioned techniques are based on an 18- to 24-hour incubation period. A variety of “rapid methods” are available as well (Jorgensen 1991). These are based on a determination of changes in bacterial growth rates caused by antimicrobial agents and can provide susceptibility in 4 to 8 hours.

Another test is to determine the serum bactericidal activity. This test is performed in a manner similar to that of MBC, except it is a serum sample from a patient treated with drugs or a combination thereof that is diluted in 2-fold steps. After a standard inoculum of the organism has been added and the mixture incubated at 35° C. for 18 hours, a small sample is subcultured onto blood agar plates, and the serum dilution that kills 99.9% of the organisms is determined.

One may also conduct in vivo studies on various bacterial species using one or more drugs (an antibiotic and a drug that is found to modulate MgrA function).

4. In vivo Antimicrobial Therapy

It is also contemplated that compounds of the present invention may be administered to a subject suffering from an infectious organism. Such embodiments may include treatment of a bacterial infection using an MgrA inhibitor to reduce virulence of the bacterium, or an agent that increases or stabilizes the interaction of MgrA with target DNA, so as to reduce bacterial resistance to antibiotics. In addition, one may also use an inhibitor of MgrA binding to DNA in conjunction with both an antibiotic and an antivirulence agent, such as RIP.

Classes of antibiotics that may be used in conjunction with compounds of the present invention include, but are not limited to, macrolides (e.g., erythromycin), penicillins (e.g., nafeillin), cephalosporins (e.g., cefazolin), carbepenems (e.g., imipenem, aztreonam), other beta-lactam antibiotics, beta-lactam inhibitors (e.g., sulbactam), oxalines (e.g., linezolid), ATP synthase inhibitors (e.g. diarylquinoline compounds, R207910), aminoglycosides (e.g., gentamicin), chloramphenicol, sufonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, polyenes (e.g., amphotericin B), rifamycins (e.g., rifampin), and azoles (e.g., fluconazole).

Examples of specific antibiotics that may be used include, but are not limited to, nafcillin, methicillin, oxacillin, cloxacillin, dicloxacillin, ampicillin, amoxicillin, carbenicillin, ticarcillin, mezlocillin, piperacillin, erythromycin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, levofloxacin, grepafloxacin, norfloxacin, nalidixic acid, sparfloxacin, pefloxacin, amifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, R207910 and nystatin.

A. Drug Formulations and Routes for Administration to Patients

Pharmaceutical compositions of the present invention comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof, a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

B. Combination Therapy

As discussed above, in order to increase the effectiveness of an antibiotic or antivirulence agent, the compounds of the present invention may be combined with traditional drugs. It is contemplated that this type of combination therapy may be used in vitro or in vivo. An “antibiotic” is capable of inhibiting the growth of microorganisms without damage to the host or killing the microorganism without damage to the host. For example, the antibiotic may inhibit cell wall synthesis, protein synthesis, nucleic acid synthesis, or alter cell membrane function. Antibiotics agents include, for example, cell-wall synthesis inhibitors (e.g., beta-lactams, glycopeptides), protein synthesis inhibitors (e.g., aminoglycosides, tetracyclines, macrolides, lincosamides), folate antagonists (e.g., sulfonamides, trimethoprim), cell-membrane inhibitors (e.g., polymyxin, colistin) and nucleic acid inhibitors (e.g., quinolones, rifampin, nitrofurantonin). An “antivirulence agent” is capable of inhibiting the virulence mechanisms of a microorganism without damage to the host thus allowing the host's immune system to kill the invading microorganism. For example, the antivirulence agent may inhibit virulence gene expression, virulence factor maturation or virulence factor trafficking inside or outside of the microorganism. Antivirulence agents include, for example, quorum sensing inhibitors (e.g. RNAIII inhibiting peptide (RIP)) and sortase enzyme inhibitors (e.g. flavonols).

More generally, agents of the present invention would be provided in a combined amount with an effective amount of an antibiotic to kill or inhibit proliferation and/or an effective amount of an antivirulence agent to inhibit the infectivity of an infectious organism, e.g., a bacterial cell. This process may involve contacting the cell(s) with the agents at the same time or within a period of time wherein separate administration of the two substances produces a desired therapeutic benefit. This may be achieved by contacting the cell, tissue or organism with a single composition or pharmacological formulation that includes both agents, or by contacting the cell with two or more distinct compositions or formulations, wherein one composition includes one agent the other includes another.

The terms “contacted” and “exposed,” when applied to a cell, tissue or organism, are used herein to describe the process by which a therapeutic agent, such as for example an antimicrobial agent, e.g., antibiotic or antivirulence agent, are delivered to a target cell, tissue or organism or are placed in direct juxtaposition with the target cell, tissue or organism. To achieve cell killing or cell growth inhibition or inhibition of infectivity, the agents are delivered to one or more cells in a combined amount effective to kill the cells, prevent them from dividing, or lessen their overall virulence.

The compounds of the present invention may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the candidate substance. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the candidate substance.

Various combination regimens of the agents may be employed. Non-limiting examples of such combinations are shown below, wherein a MgrA or MgrA homolog modulator is “A” and a second agent, such as an antibiotic or an antivirulence agent, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Functional Studies

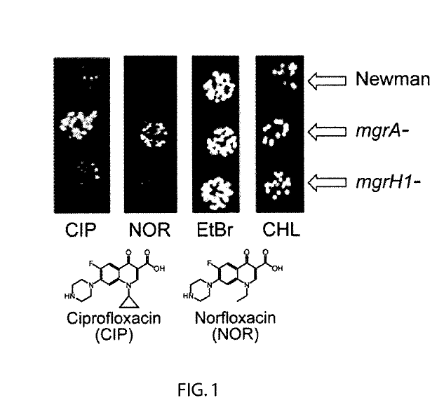

Regulation of antibiotic resistance by MgrA. The inventors were intrigued by the mechanism used by MarR family proteins to regulate antibiotic resistance (FIG. 1), and thus decided to study S. aureus instead of E. coli because of the medical importance of this microorganism. A sequence search in the S. aureus genome was performed and identified MgrA and MgrH1 as homologues of E. coli MarR. To evaluate the function of these proteins, the inventors obtained the insertion knock out mutant strains mgrA- and mgrH1- of the S. aureus Newman strain (Bae et al., 2004). Antibiotic resistance tests of these strains were performed.

S. aureus cells were serially diluted and an equal number of cells was spotted on a plate containing various concentrations of antibiotics. The S. aureus strain Newman (wild-type) and the mutant strains mgrA- and mgrH1- (knock outs) were tested. While the mgrH1- strain did not show any phenotype, the mgrA- strain showed higher resistance than the Newman strain to the fluoroquinolone antibiotics ciprofloxacin (CIP) and norfloxacin (NOR; FIG. 1). A further study revealed that the mgrA- strain is more resistant to vancomycin (VCM) as well. The MIC measurement results are shown in Table 1. The data indicate that the mgrA- strain exhibits medium-level resistance compared to the wild-type.

TABLE 1
Susceptibilities of Strains to Fluoroquinolones and Vancomycin

| | | MIC (μg ml⁻¹) | |
|---|---|---|---|
| Strain | CIP | NOR | VCM |
| Wild-type | 0.35 | 0.7 | 1.2 |
| (Newman) | | | |
| mgrA mutant | 0.7 | 1.4 | 2.4 |
| (mgrA⁻) | | | |

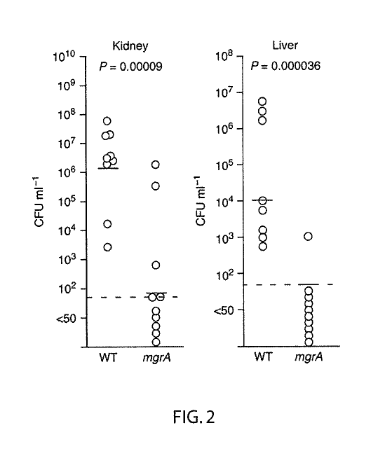

MgrA is a major virulence determinant. Next, using the murine abscess model (Bae et al., 2004), the inventors investigated whether MgrA contributes to the pathogenicity of S. aureus. Bacteria (10⁶ cfu for each strain) were injected intravenously into ten mice, and the animals were sacrificed 4 days after formation of infection. The kidneys and liver were harvested and incubated on agar medium for S. aureus colony formation and enumeration. As can be seen in FIG. 2, the mgrA- strain shows a 10,000-fold (4-log) reduction of colony forming units in kidneys, and at least a 100-fold (2-log) reduction in liver compared to the wild-type S. aureus Newman strain. The experiment was repeated and similar results were obtained. This major reduction of virulence observed for the mgrA- knock out strain is unprecedented in regulators studied in S. aureus. It shows that MgrA is a major virulence determinant and an ideal target for the prevention and treatment of S. aureus infection.

### Example 2

### Crystallization Studies

MgrA expression and crystallization. Since MgrA plays a major role in virulence regulation in S. aureus, the inventors decided to structurally characterize this protein in the hope of gaining hints into its regulatory mechanism. The wild-type MgrA was cloned into pET28a with an N-terminal His₆-tag and expressed with BL21-(DE3)-competent E. coli cells. The protein was purified by Ni²⁺-His trap column, the His₆-tag was cleaved, and the resulting protein was further purified by Mono S column. This wild-type MgrA, bearing Gly and Ser residues at the N-terminus due to cleavage of the His6-tag with thrombin, was successfully crystallized in 1.6 M ammonium sulfate, 0.1 M MES (pH 6.0). However, these crystals only diffracted to 3.5 Å despite efforts to improve their quality. Sequence alignment of MgrA with the MarR family proteins indicated that the first five and last four residues of MgrA are not highly conserved and are most likely unstructured. Thus, the inventors prepared a mutant MgrAΔ (MgrA1,5-143) with deletion of the second to fifth and last four residues of the protein. The truncated protein was overexpressed, purified and crystallized under the same conditions as the wild-type. Crystals from the truncated MgrAΔ diffracted to 2.85 Å, a significant improvement over the wild-type. Selenomethionine-substituted MgrAΔ was also prepared and crystallized. This structure was solved by multiwavelength anomalous dispersion (MAD) and represents the first crystal structure of MgrA.

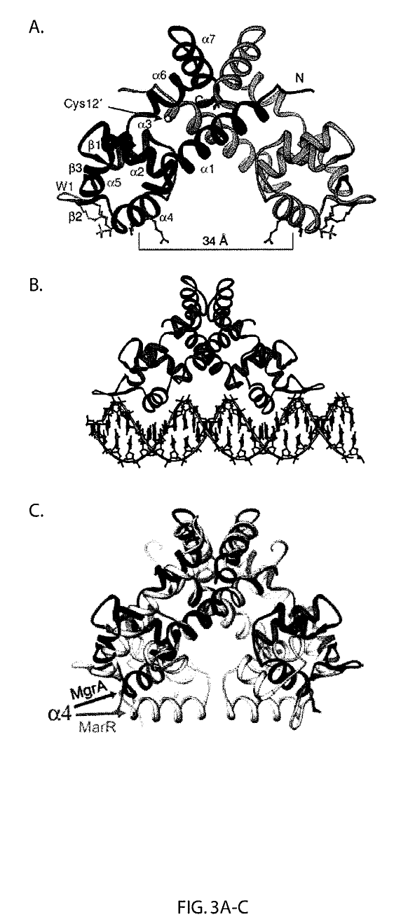

Overall structure of MgrA. The overall structure of MgrA resembles that of E. coli MarR and its homologues (FIGS. 3A-C). It consists of a dimer related by a crystallographic two-fold rotation (FIG. 3A). The dimer is rich with a helices and is triangular in shape with two winged helix DNA binding domains (β1-α3-α4-β2-W1-β3, FIGS. 3A-C). This DNA binding domain is connected to the N- and C-terminal dimerization domain through helices α1, α5 and α6. The dimerization domain consists of helices α1, α6 and α7 with hydrophobic residues buried at the interface. The long helix (α5) that connects the DNA binding domain with the C-terminal dimerization domain in E. coli MarR is separated into two helices, α5 and α6, in MgrA. The structure of the MgrA dimer shows significant differences from the E. coli MarR dimer containing bound salicylates. Most noticeably, the putative small molecule binding pockets observed in E. coli MarR could not be found in MgrA.

The DNA binding domains of the MgrA structure also adopt a different conformation from those of MarR (FIG. 3C). The two DNA-binding helices (α4) in the MgrA dimer are orientated parallel to each other and separated by 34 Å. This spacing allows each α4 helix to be buried in the major groove one turn apart in a continuous DNA duplex (FIG. 3B, molecular model of the MgrA-DNA complex was generated by manually docking MgrA onto an ideal model of the operator DNA). The wing section and the N-terminus of α2 can also interact with the DNA backbone and/or minor groove in this model. Several basic residues protrude out of the proposed DNA-binding surface. The sulfate anion overlaps with the duplex DNA backbone in the complex model (FIG. 3B), which suggests that these basic residues interact with the DNA backbone. From these analyses, the inventors concluded that the apo-MgrA dimer adopts an optimum conformation to interact with DNA.

### Example 3

### Oxidation of MgrA Cys

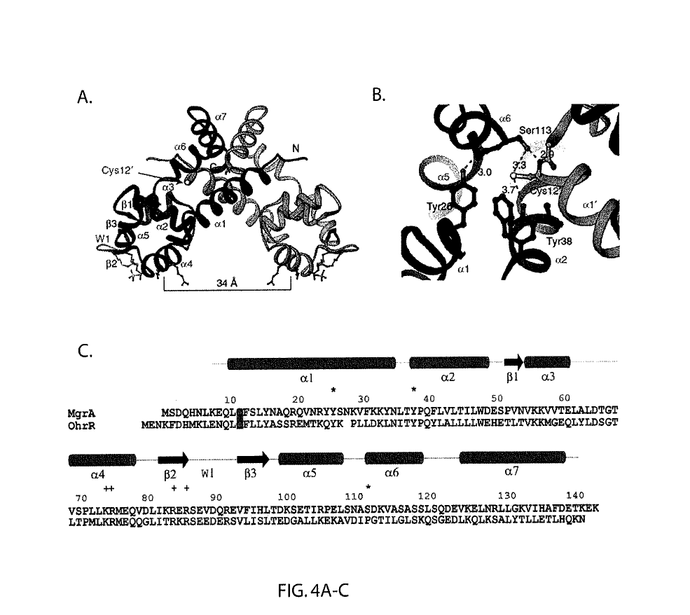

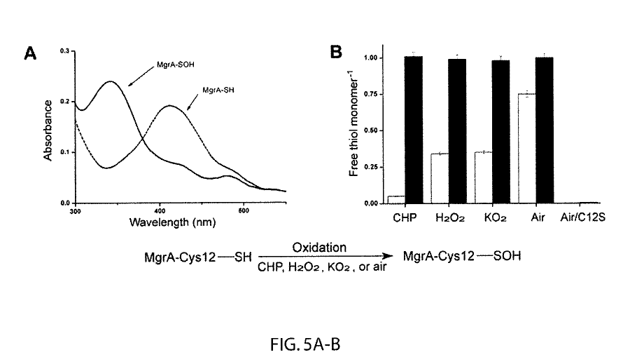

The regulation mechanism of MgrA. The structure of MgrA, when it was first solved, did not provide obvious insights into the mechanism of this protein. The inventors were very intrigued by what they had learned about MgrA:
i) MgrA is a DNA binding protein based on its structure and gel shift experiments.
ii) MgrA regulates S. aureus responses to fluoroquinolones (CIP and NOR, FIG. 1), vancomycin, and oxacillin (data not shown). How can MgrA, a protein with 147 aa, recognize three completely different organic structures?
iii) Binding assays indicate MgrA does not bind fluoroquinolones or vancomycin.
iv) The transcription profiling study shows that MgrA regulates 350 genes in S. aureus; however, most of these genes are not related to antibiotic resistance. Therefore, MgrA must sense some other host or environmental signals.
   The mechanistic issue for MgrA frustrated the inventors after they obtained the first crystal structure of this protein. Numerous binding assays were peformed to test a variety of hypotheses. A breakthrough came from a careful inspection of the MgrA structure and its sequence, when it was noticed that there is only one Cys residue present in the entire MgrA sequence, at position 12. This Cys residue, located in the N-terminal α1, is precisely in the dimerization domain (FIG. 4A) and seems to play an important role in orienting the MgrA dimer conformation. Each monomer presents its Cys12 to be recognized through hydrogen bonding to Ser113 and Tyr38 from the other monomer (FIG. 4B).

The presence of a unique Cys residue in the dimerization interface of MgrA reminded the inventors of another MarR family protein, OhrR. OhrR is a peroxide-sensing transcriptional factor in B. subtilis, which negatively regulates a peroxide-resistance gene, ohrA (Fuangthong et al., 2001; Fuangthong and Helmann, 2002; Hong et al., 2005; Mongkolsuk et al., 1998). Sequence alignment shows that OhrR and MgrA share significant sequence homology (FIG. 4C). The lone Cys residue in OhrR, Cys15, also lies in the interface of the OhrR dimer and is recognized by residues from the other monomer (Hong et al., 2005). It has been shown that oxidation of OhrR by hydrogen peroxide or organic hydroperoxides leads to dissociation of OhrR from the promoter DNA and activation of the ohrA gene. The regulatory mechanism involves oxidation of Cys15 to form sulphenic acid (demonstrated in vitro), which disrupts the hydrogen bonding network and changes the conformation of the dimer (Fuangthong and Helmann, 2002; Hong et al., 2005). The sulphenic acid product is likely further modified in vivo by reducing agents inside B. subtilis. The structure of the oxidized OhrR has not been obtained; however, evidence from in vitro and in vivo investigations have firmly established the Cys oxidation mechanism. Organic hydroperoxides possessing hydrophobic groups are much more effective than hydrogen peroxide in oxidizing Cys 15. The presence of a hydrophobic pocket around Cys15 explains this preference (Hong et al., 2005). A similar hydrophobic pocket can also be identified in the MgrA structure.

The sequence and structural similarities between MgrA and OhrR strongly suggest that MgrA is also an oxidation sensor. These similarities include the conservation of the lone Cys residue in the dimerization interface, the presence of a similar hydrogen-bonding network, and the hydrophobic pocket around the Cys residue. OhrR is known to regulate only one gene in B. subtilis to counter peroxide stress. MgrA Cys12 could be oxidized by reactive oxygen species (ROS), which could turn on its broad regulatory function.

Oxidation of MgrA. To test the oxidation sensing hypothesis, the inventors isolated MgrA under reducing conditions, removed the reducing agent through buffer exchange at 4° C., and treated 100 μl of reduced MgrA (50 μM) with 4 equivalents of cumene hydroperoxide (CHP, an organic hydroperoxide), H₂O₂, or KO₂ at room temperature for 10 minutes. Cys12 oxidation to Cys-sulphenic acid was confirmed by trapping the oxidized MgrA with 4-chloro-7-nitrobenzo-2-oxa-1,3-diazole (NBD-Cl), the product of which absorbs at 347 nm (Ellis and Poole, 1997). The reduced protein reacts with NBD-Cl to form a thiol-NBD conjugate with an absorbance maximum at 420 nm (FIG. 5A) (Ellis and Poole, 1997). Since Cys12 is the lone Cys residue, one can quantitatively evaluate its oxidation by measuring the free thiol contents per MgrA monomer with the DTNB (5,5′-dithiobis(2-nitrobenzoic acid)) assay (Riddles et al., 1983). As shown in FIG. 5B, cumene hydroperoxide, hydrogen peroxide, and superoxide are all capable of oxidizing the free thiol of Cys12 in MgrA. Cumene hydroperoxide appears to be the most efficient oxidant in this group. After 10 minutes treatment with 4 equivalents of CHP, over 90% of Cys12 was oxidized to sulphenic acid. The hydrophobic residues near Cys12 in the MgrA structure may contribute to the preferred oxidation of Cys12 by CHP over hydrogen peroxide. This result also implies that small molecules could be developed to selectively target this pocket around Cys12. A C12S mutant with Cys12 mutated to Ser12 was used as the control. No thiol group was observed with this mutant protein.

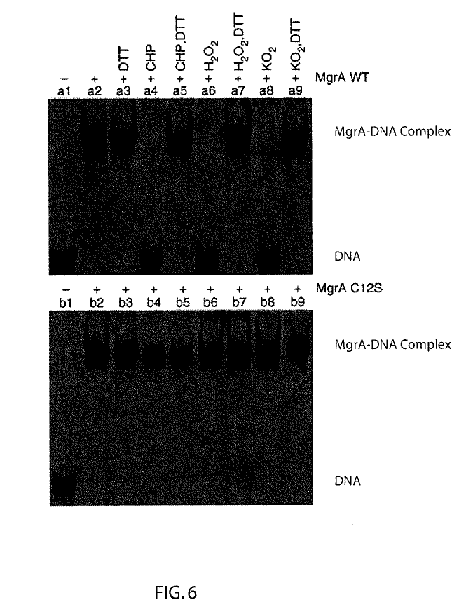

Oxidation of MgrA leads to its dissociation from promoter DNA. The inventors have shown that Cys12 in MgrA is prone to oxidation by peroxides and superoxides. To determine whether this finding is biologically relevant, they evaluated whether oxidation of Cys12 affects MgrA's binding to DNA. The inventors performed electrophoretic mobility shift experiments using the promoter sequence of sarV that has been characterized as interacting with MgrA (FIG. 6) (Manna et al., 2004). Addition of excess amounts of MgrA to the DNA probe led to formation of the MgrA-DNA complex. Oxidation of MgrA with different oxidants dissociated the protein from DNA (FIG. 6). Treating the oxidized MgrA with a reducing agent (DTT) can regenerate the MgrA-DNA complex. The MgrAC12S mutant was used as the control. This mutant protein remained bound to DNA under the same oxidation conditions that disrupt the wild-type MgrA-DNA interaction. The result firmly established the mechanism that oxidation of Cys12 leads to dissociation of MgrA from DNA.

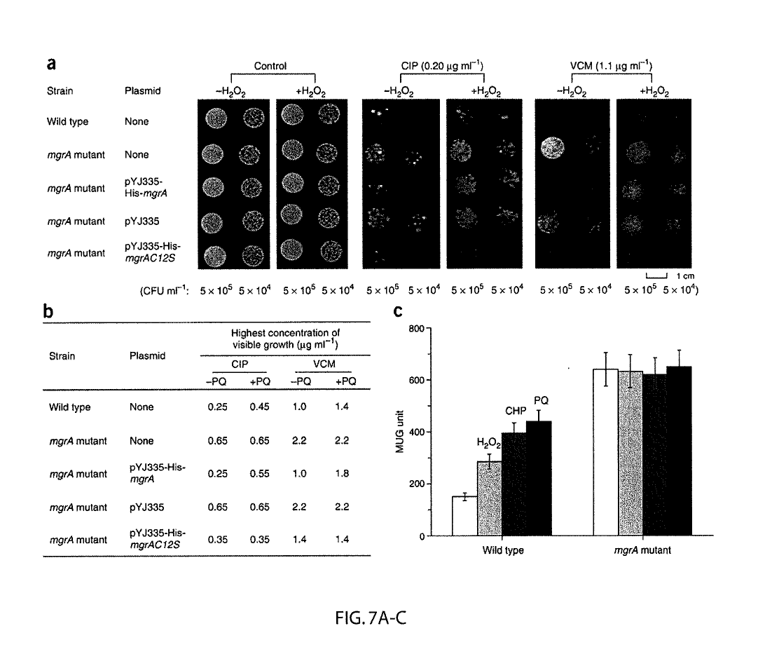

MgrA oxidation in vivo. To further confirm the oxidation sensing mechanism for MgrA in vivo, the inventors first chose antibiotic resistance as the readout for the MgrA-based regulation. If MgrA dissociates from DNA upon sensing a signal, increased resistance toward fluoroquinolones and vancomycin should be observed in S. aureus. Five strains were used to test oxidation responses in vivo: the wild-type Newman, the mgrA- mutant (knock out), the mgrA- mutant complemented with mgrA cloned in a plasmid pYJ335, the mgrA- mutant complemented with mgrAC12S in pYJ335, and the control mutant strain with pYJ335. As shown in FIG. 7A, all strains grew without noticeable differences under normal conditions. The mgrA- mutant strain and the mutant strain with plasmid pYJ335 exhibited resistance towards antibiotics CIP and VCM (FIGS. 1 and 7A-B), while the Newman, the mgrA- mutant strain complemented with pYJ335-His-mgrA, and the mgrA- mutant strain complemented with pYJ335-His-mgrAC12S were susceptible toward these antibiotics under the same growth conditions. Importantly, when treated with 100 μM H₂O₂, the Newman strain and the mgrA- mutant strain carrying pYJ335-His-mgrA exhibited increased resistance comparable to that of the mgrA- mutant strain. This result showed that, in S. aureus strains expressing MgrA, oxidation of MgrA in vivo leads to its dissociation from DNA and subsequent activation of antibiotic resistance.

The mgrA- mutant strain carrying pYJ335-His-mgrAC12S was used as a control. This strain did not show a noticeable change under oxidation conditions as compared to that without H₂O₂. The reason for the higher level of drug resistance observed for this strain than the Newman strain is currently unclear. The inventors suspect that the MgrAC12S mutant protein is unstable and has a weaker affinity to DNA than the wild-type MgrA.

The susceptibility levels of all five strains toward CIP and VCM were also evaluated in the absence and presence of paraquat (PQ), another reagent known to cause oxidative stress in vivo (FIG. 7B). The result clearly demonstrated that the Newman strain and the mgrA- mutant strain complemented with pYJ335-His-mgrA exhibit increased antibiotic resistance in the presence of PQ, indicating again that oxidation of MgrA activates the antibiotic resistance pathways. The levels of antibiotic resistance observed here are medium levels (inherent resistance instead of acquired resistance) and may not be the same as those seen clinically.

Lastly, the inventors constructed a norA-lacZ reporter fusion in the chromosomes of the wild-type and mgrA- mutant strains (norA encodes a multidrug efflux transporter protein responsible for resistance to fluoroquinolones; this gene is regulated by mgrA in S. aureus) (Truong-Bolduc et al., 2003). β-Galactosidase activity of these two strains was determined in the presence and absence of oxidation challenge (FIG. 7C, with H₂O₂, CHP, or PQ, respectively). The mgrA- mutant strain (knock out) showed a relatively high level of norA induction under all conditions. Induction of norA was repressed in the wild-type Newman strain in the absence of oxidation challenge; however, a consistent increase (2-3 folds) of norA induction was observed under the oxidation conditions tested. This in vivo result further confirms the proposed oxidation regulation mechanism for MgrA.

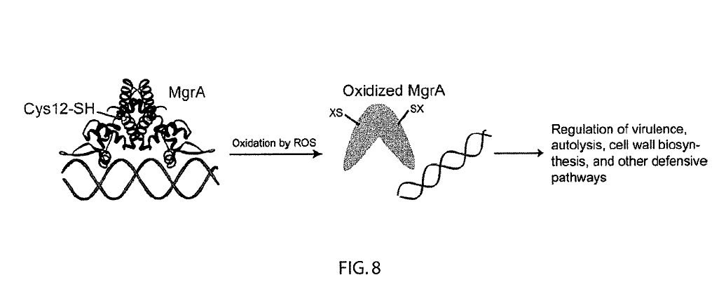

Summary. This study of MgrA revealed an oxidation sensing mechanism used to regulate gene activation in S. aureus (FIG. 8). Mechanistically, this is very similar to that of OhrR in B. subtilis. However, while OhrR only regulates one peroxide-resistance gene, MgrA regulates many genes that perform a range of diverse functions. What is the link between the oxidation sensing mechanism of MgrA and its regulatory functions? It turns out the most effective response from the immune system to defend against infections by Gram-positive pathogens such as S. aureus is to generate reactive oxygen and nitrogen species to counter invading microbes (Rooijakkers et al., 2005; Maruyama et al., 2003). Apparently, S. aureus has responded to this challenge from the host by adopting an oxidation sensing mechanism to sense the immune response and activate a range of defensive pathways (FIG. 8) leading to thickening of its cell wall, modifications of its metabolic pathways, and a reduction in virulence. It is known that antibiotics such as fluoroquinolones and vancomycin can induce oxidative stress as a side effect. Thus, the mgrA regulon in S. aureus also includes a few antibiotic resistance genes as an inherent defensive measure. The oxidation sensing regulation used by MgrA is simple but effective and could be used by many other regulators in S. aureus or by other pathogens.

This discovery opens doors for tuning these global regulators with small molecules to treat infections. Since the hydrogen-bonding network around Cys12 plays a major role in MgrA's function, the inventors envision disrupting these interactions with small molecules. Specifically, Cys12 can be alkylated with selective alkylators recognizing the hydrophobic pocket near this residue. The small molecule would work as a surrogate oxidative stress signal, and the alkylation would lead to dissociation of MgrA from DNA and suppression of S. aureus virulence.

### Example 4

### MgrH1 and SarA

MgrH1 is a close homolog of MgrA and OhrR. The lone Cys residue, the residues that form the hydrogen bonding network, and the residues that form the hydrophobic pocket around the Cys residue are all conserved in the sequence by MgrH1. Once the inventors discovered the major role played by MgrA in S. aureus, they started to wonder about the function of MgrH1. They tested potential virulence regulation of MgrH1 with a murine abscess model system by comparing the mgrH1- strain and the wild-type Newman strain. A 20-fold reduction of virulence in liver was obtained from the mgrH1- strain compared to the Newman strain (Table 2). Preliminary work on the mgrH1- mutant strain also revealed that it possesses resistance to vancomycin and has a different autolytic behavior from the Newman strain. They cloned, expressed, and purified MgrH1 and tested oxidation of Cys13 by various oxidants. Preliminary data indicate that Cys13 in MgrH1 is more sensitive to peroxide oxidation than Cys12 in MgrA (Table 2).

TABLE 2
Comparison of MgrA, MgrH1 and SarA

| | | | Virulence |
|---|---|---|---|
| | | | regulation |
| Protein | Oxidation by H₂O₂ | Oxidation by CHP | (knock out) |
| MgrA | 4 equiv, 10 min, 65% | 4 equiv, 10 min, 95% | 10,000-fold |
| | oxidation at r.t. | oxidation at r.t. | reduction in |
| | | | kidney 100- |
| | | | fold reduction |
| | | | in liver |
| MgrH1 | 4 equiv, 10 min, 71% | 2 equiv, 10 min, | 20-fold |
| | oxidation at r.t. | 100% oxidation at r.t. | reduction in |
| | | | liver |
| SarA | 100 equiv, 30 min, | 100 equiv, 30 min, | Reduction of |
| | 15% oxidation at r.t. | ˜50% oxidation at | virulence was |
| | | 37° C. | suggested |

To further elucidate the function of MgrH1, transcription profiling of the mgrH1 regulon in S. aureus was conducted (Truong-Bolduc et al., 2005; Dunman et al., 2001). In total, 12 chip experiments were performed on the wild-type Newman strain and mgrH1- mutant strain after 2 h and 5 h growth at 37° C. (each condition was repeated three times). The microarray result suggested that MgrH1 regulates ˜80 genes in S. aureus. These genes include virulence factors such as exotoxins, surface proteins, autolysis factors, and genes involved in metabolic switching, antibiotic resistance, and peroxide resistance. It appears that the mgrH1 regulons complement defensive functions of the mgrA regulon.

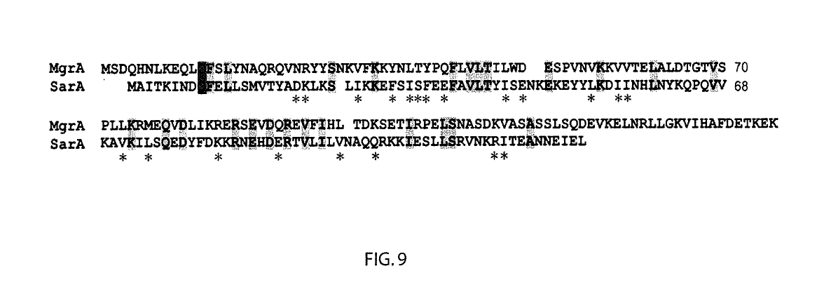

Among all the MarR type regulators in S. aureus, the unique Cys residue is found in three proteins, MgrA, MgrH1, and SarA. The inventors have shown that MgrA and MgrH1 are highly homologous in sequence and quite susceptible to oxidation. SarA is less homologous to MgrA but possesses a lone Cys residue, Cys 9 (FIG. 9). A previous random mutagenesis study has identified this Cys residue to be important in the SarA- based regulation (Liu et al., 2006). Expression of a SarAC9A mutant protein in the sarA- mutant strain led to activation of sara, spa, and aur, all regulated by SarA. No activation was observed in a control experiment with the wild-type SarA.

SarA may adopt an oxidation sensing mechanism similar to MgrA. Cys9 in SarA might be oxidized by ROS or other reactive species which could lead to its dissociation from DNA and activation of the sarA regulon. To test this hypothesis, they cloned, expressed, and purified SarA. The Cys9 residue of SarA was found to be oxidized by CHP and hydrogen peroxide; however, it took more oxidants and a longer time to oxidize Cys9 in SarA than those for MgrA and MgrH1 (Table 2).

To monitor oxidation induced dissociation of SarA from DNA, the inventors conducted fluorescence anisotropy (FA) experiments with a fluorescein-labeled DNA that is known to be recognized by SarA. FIG. 10A clearly showed that upon binding to SarA, the complex gave a higher FA readout. Upon treating with CHP, SarA dissociated from DNA. In a control experiment with the C9S mutant SarA, CHP did not lead to a significant change of FA (FIG. 10B). The result confirmed the oxidation dissociation mechanism of SarA. The FA method also provides a way to monitor binding of MgrA, MgrH1, or SarA to DNA.

The inventors also monitored activation of a virulence gene splB in S. aureus. This gene is known to be repressed by SarA. As shown from qRT-PCR experiments (FIG. 10C), activation of this gene was repressed in the Newman strain. The sarA mutant strain exhibited a higher activation level of this gene. When the Newman strain was treated with 0.4 mM H₂O₂ for 30 min, the splB gene was activated by oxidation, confirming the in vivo oxidation sensing proposed for SarA.

### Example 5

### Small Molecule Tuning of MgrA

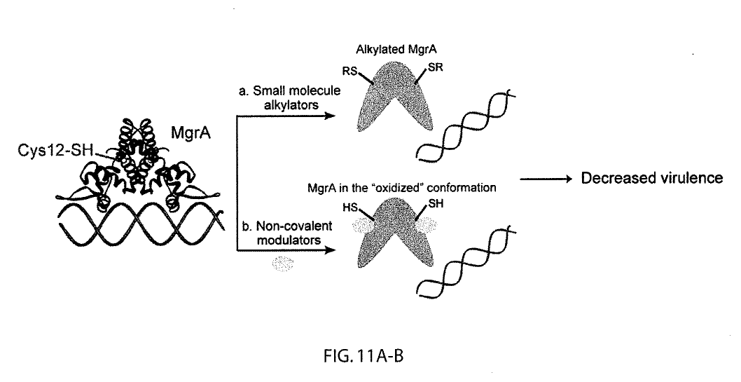

The general strategy to modulate MgrA's function is summarized in FIG. 11. In a covalent modification strategy (strategy A in FIG. 11), the inventors plan to disrupt the hydrogen-bonding network of Cys12 by selective alkylation of this residue. This modification would lead to dissociation of MgrA from DNA and subsequent down regulation of virulence factors in S. aureus. This strategy is very attractive due to the extensive knowledge of irreversible alkylation of Cys residues in Cys-based proteases (Otto, 1997; Powers et al., 2002; Leung and Fairlie, 2000). One can borrow the concepts and chemistry developed from decades of research. Several groups of molecules identified in previous work show selectivity towards Cys residues in Cys-based proteases and have low toxicity. A non-covalent strategy (strategy B in FIG. 11) can be employed as well. Presumably, a small molecule may bind MgrA and stabilize its “oxidized” conformation, reducing virulence of bacterial cells.

Irreversible inhibition of Cys proteases have been demonstrated with many alkylating agents, such as halomethyl ketones, diazomethyl ketones, acyloxymethyl ketones, epoxides, and vinyl sulfones (Powers et al., 2002). These agents alkyate the catalytic Cys residues and render them inactive. Due to the high toxicity most of these agents have no potential in medicine. Two exceptions include epoxides and vinyl sulfones (Powers et al., 2002). These agents are generally less reactive and have very low toxicity (Palmer et al., 1995). They have been incorporated as “warheads” to selectively target specific Cys residues in proteases. In fact, a study has indicated that phenyl vinyl sulfone can efficiently alkylate Cys12.

Alkylation of Cys12 in MgrA in vitro and in vivo. In a study to test potential alkylation of Cys12 in MgrA, the inventors examined several alkylators (1-7, Table 3). Eight equivalents of alkylators were used to react with 0.05 mM MgrA in water for 1 h at pH 7.4 at room temperature. Alkylation of Cys12 was determined by DTNB assay for free thiol content. The results clearly showed that Cys 12 can be alkylated with a range of different agents (Table 3). Comparison of the first three reactions showed that addition of hydrophobic groups can enhance the activity and, presumably, selectivity of the alkylators, as the simple alkylator 1 has lower activity than 2 or 3. A vinyl sulfone (7) exhibited one of the best results. Over 95% of cysteine thiols were alkylated by compound 7 after 1 h of reaction.

TABLE 3
Preliminary results on Cys12 alkylation

| Alkyation reagent | | Condition | Potential product | | Alkylation percentage |
|---|---|---|---|---|---|
| 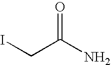 | 1 | 1 h, r.t. | 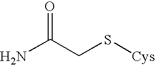 | 1b | 62% |
| 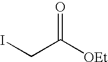 | 2 | 1 h, r.t. | 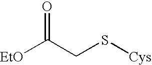 | 2b | >95% |
| 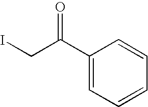 | 3 | 1 h, r.t. | 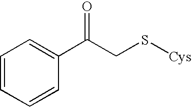 | 3b | >95% |
| 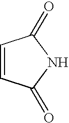 | 4 | 1 h, r.t. | 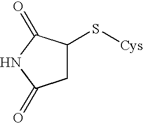 | 4b | >95% |
| 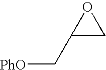 | 5 | 1 h, r.t. | 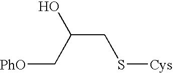 | 5b | 48% |
| 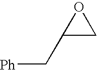 | 6 | 1 h, r.t. | 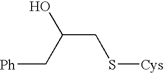 | 6b | 59% |
| 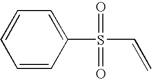 | 7 | 1 h, r.t. | 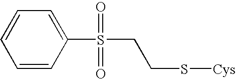 | 7b | >95% |

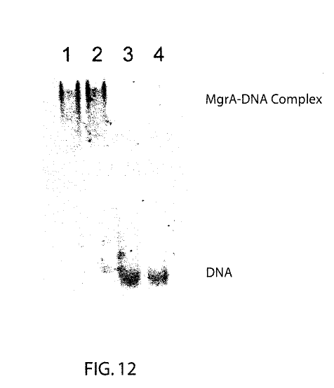

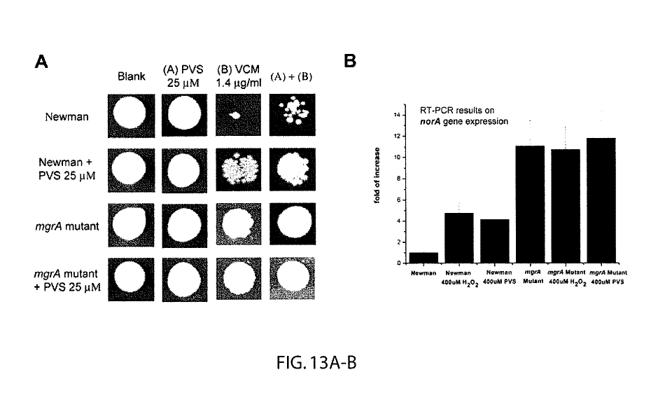

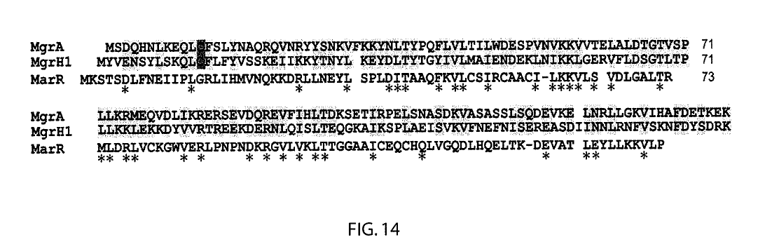

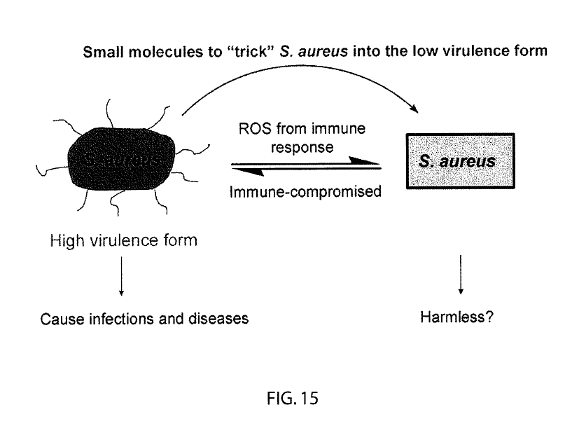

To validate the proposed strategy that alkylation of Cys12 would change MgrA's conformation and dissociate this protein from DNA, a preliminary gel shift experiment was performed as shown in FIG. 12. The apo-MgrA binds the promoter DNA, while alkylation of Cys12 leads to dissociation from DNA. The alkylation reactions were run at 37° C., room temperature, and 4° C. (overnight) with the same results obtained. Control experiments with MgrAC12S mutant showed no change in DNA affinity in the presence of alkylators. Thus, the observed dissociation of alkylated MgrA is due to modification of the Cys12 residue. Preliminary studies also showed that the alkylated MgrA exhibits similar chromatographic properties to the wild-type MgrA. Protein denaturing by alkylation of Cys12 should not account for the observed dissociation of MgrA from DNA. Even if alkylation of Cys12 led to denaturing of the protein, the goal of modulating this protein's function was achieved.

Activation of MgrA by phenyl vinyl sulfone (PVS) inside S. aureus was tested. In the presence of 25 μM PVS, the Newman strain exhibited an enhanced resistance to vancomycin (FIG. 13A). This result showed that PVS can react with MgrA and activate the mgrA operon via alkylation. To further confirm the effect of PVS, the inventors monitored activation of the norA gene known to be repressed by MgrA. As shown by qRT-PCR experiments (FIG. 13B), PVS activated norA as efficient as H₂O₂, clearly showing that alkylation of MgrA can turn on the mgrA operon. The next challenge is to develop vinyl sulfones that have high affinity and specificity toward MgrA. Of course, a non-covalent modulator that can activate MgrA with high sensitivity and specificity via direct binding is also highly desirable.

A library of vinyl sulfones may be prepared and tested for their activities in vitro and in vivo (including animal experiments). The non-covalent idea suggested in FIG. 12, strategy B, may also be tested. A small molecule may bind to MgrA and stabilize it in the “oxidized” conformation without covalent modification of the protein. These molecules may suppress the virulence of S. aureus.

### Example 6

### P. aeruginosa homologues

P. aeruginosa. There are two close MgrA homologues (functions have never been reported) in P. aeruginosa:
mstrgkvarp dgveesllld nqlcfklyaa sravirgyrp lleqigltyp qylvmlvlwe whasppeqpt vkalgdrlll dsgtltpllk rleqlglvdr rrashderev hltltvpgit lrerviplrq qlicstgfdl nemfdlhqrl ggllsrfrlv vgg and
msrlpttpce qlkldnqlcf alystslqmt kvykpllqtl gltypqyiam lvlwerdglt vgeisarmlt dpgsltpllk rlegeglitr trssedervv llrltdkgra lqrqaesipa cilestgldl pqlvalkdel lalrgnlqap d
   Both proteins have the conserved lone Cys residue that could be oxidized by reactive oxygen or nitrogen species like MgrA. One or both of these proteins could regulate the virulence of the pathogen as MgrA does in S. aureus. The inventors propose these two proteins as targets for suppressing virulence of P. aeruginosa. The covalent and non-covalent strategies (FIG. 11, strategies a and b) can be applied to tune their function with small molecules.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

6. References

The following references, to the extent that they provide exemplary procedural or other details supplementary to those set forth herein, are specifically incorporated herein by reference:
Archer et al., Antimicrob. Agents Chemother., 34:1720-1724, 1990.
Bae et al., Proc. Natl. Acad. Sci. USA, 101:12312-12317, 2004.
Berger-Bachi, Trends Microbiol., 2:389-393, 1994.
Cheung et al., FEMS Immunol. Med. Microbiol., 40:1-9, 2004.
Chougnet et al., Bioorg. Med. Chem. Lett., 13:3643-3645, 2003.
Donato et al., Drug Metab. Dispos., 32:699-706, 2004.
Dunman et al., J. Bacteriol., 183:7341-7353, 2001.
Dunn et al., J. Am. Chem. Soc., 124:10254-10255, 2002.
Ellis and Poole, Biochemistry, 36:15013-15018, 1997.
Fuangthong and Helmann, Proc. Natl. Acad. Sci. USA, 99:6690-6695, 2002.
Fuangthong et al., J. Bacteriol., 183:4134-4141, 2001.
Geha, J. Clin. Microbiol., 32:1768-1772, 1994.
Hong etal., Mol. Cell, 20:131-141,2005.
Ingavale et al., Mol. Microbiol. 48, 1451-1466, 2003.
Jorgensen, J. Chemother., 3(1):155-157, 1991
Kaatz et al., Antimicrob Agents Chemother. 49(1):161-9, 2005.
Kaatz et al., J. Infect. Dis., 163:1080, 1991.
Kinsman et al., Pathology, 66:325, 1985.
Kobayashi et al., Biochemistry, 37:6679-6688, 1998.
Leung and Fairlie, J. Med. Chem., 43:305-341, 2000.
Liu et al., Arch. Biochem. Biophys., 424:33-43, 2004.
Liu et al., Proc. Natl. Acad. Sci. USA, 103:2392-2397, 2006.
Luong et al., J. Bacteriol. 185(13):3703-10, 2003.
Luong et al., Bacteriol. 188, 1899-1910, 2006.
Manna et al., J. Bacteriol., 186:5267-5280, 2004.
Maranan et al., Infect. Dis. Clin. North Am., 11:813-849, 1997.
Maruyama et al., Microbiology, 149:389-398, 2003.
Mongkolsuk et al., J. Bacteriol., 180:2636-2643, 1998.
Moorman, et al., Antimicrob. Agents Chemother., 20:709, 1981.
Murakami and Tomasz, J. Bacteriol., 171(2):874-879. 1989
Nakamura et al., Anal. Biochem., 292:280-286, 2001.
Otto, Chem. Rev. 97:133-171, 1997.
Palmer et al., J. Med. Chem., 38:3193-3196, 1995.
Peacock et al., J. Infect. Dis., 144:575, 1981.
Powers et al., Chem. Rev., 102:4639-4750, 2002.
Remington's Pharmaceutical Sciences, 18th Ed. Mack Printing Company, pp. 1289-1329, 1990.
Riddles et al., Methods Enzymol., 91:49-60, 1983.
Rooijakkers et al., Trends. Microbiol., 13:596-601, 2005.
Roser and Thomas, Z. Naturforsch., 55:915-922, 2000.
Sierra-Madero et al., Antimicrob. Agents Chemother., 32:1754-1757, 1988.
Thapliyal and Maru, Food Chem. Toxicol., 39:541-547, 2001.
Truong-Bolduc et al., J. Bacteriol., 185:3127-3138, 2003.
Truong-Bolduc et al., J. Bacteriol., 187:2395-2405, 2005.
Udo et al., J. Med. Microbiol., 37:180, 1992.
Vaudaux, et al., J. Infect. Dis., 139:547, 1979.

What is claimed is:

1. A method of identifying a modulator of bacterial MgrA function comprising:
(a) providing an MgrA polypeptide or fragment thereof that (i) binds DNA and (ii) comprises a cysteine residue corresponding to that found at Cys12 of Staphylococcus aureus MgrA;
(b) contacting said MgrA polypeptide or fragment with a candidate substance; and
(c) assessing the binding of said MgrA polypeptide or fragment to a target DNA,
wherein a change in the binding of said MgrA polypeptide or fragment to said target DNA, as compared to binding in the absence of said candidate substance, identifies said candidate substance as a modulator of bacterial MgrA function.

2. The method of claim 1, wherein the MgrA is from a Staphylococcus species, a Bacilles species, a Mycobacterium species, a Pseudomonas or a Streptococcus species.

3. The method of claim 2, wherein the Staphylococcus species is S. aureus or S. epidermidis.

4. (canceled)

5. The method of claim 2, wherein the Bacilles species is B. anthracis or B. cereus.

6. (canceled)

7. The method of claim 2, wherein the Mycobacterium species is M. tuberculosis.

8. (canceled)

9. The method of claim 2, wherein the Pseudomonas species is P. aeruginosa.

10. (canceled)

11. The method of claim 2, wherein the Streptococcus species is S. agalactiae.

12. The method of claim 1, wherein the candidate substance is a peptide or a peptidomimetic.

13. The method of claim 1, wherein the candidate substance is an organopharmaceutical.

14. The method of claim 1, wherein MgrA polypeptide or fragment binding to DNA is measured by a gel mobility shift assay, a South-Western blot, fluorescence anisotropy or FRET assay.

15. The method of claim 1, wherein at least steps (a) and (b) are performed in a cell free system.

16. The method of claim 1, wherein at least steps (a) and (b) are performed in a bacterial cell.

17. The method of claim 1, wherein said MgrA polypeptide or fragment contains an oxidized Cys12 residue.

18. The method of claim 1, wherein said MgrA polypeptide or fragment contains a reduced Cys12 residue.

19. The method of claim 1, wherein said MgrA polypeptide or fragment contains an alkylated Cys12 residue.

20. The method of claim 1, further comprising contacting said MgrA polypeptide or fragment with an oxidizing agent or alkylating agent.

21. The method of claim 20, wherein said oxidizing agent or alkylating agent is added (i) prior to step (b) or (ii) after step (b) and before step (c).

22. The method of claim 20, wherein said oxidizing agent is hydrogen peroxide, an organic hydroperoxide, nitric oxide or superoxide.

23. The method of claim 20, wherein said alkylating agent is selected from the group consisting of an aldehyde, a halomethyl ketone, a diazomethyl ketone, an acyloxymethyl ketone, an epoxide and a vinyl sulfone.

24. A method of identifying a modulator of bacterial MgrA function comprising:
(a) providing an MgrA polypeptide or fragment thereof that (i) binds DNA and (ii) comprises a cysteine residue corresponding to that found at Cys12 of Staphylococcus aureus MgrA;
(b) contacting said MgrA polypeptide or fragment with a target DNA;
(c) contacting said MgrA polypeptide or fragment/DNA complex with a candidate substance; and
(d) assessing the release of said MgrA polypeptide or fragment from said target DNA,
wherein a change in the release of said MgrA polypeptide or fragment from said target DNA, as compared to release in the absence of said candidate substance, identifies said candidate substance as a modulator of bacterial MgrA function.

25. The method of claim 24, wherein the MgrA is from a Staphylococcus species, a Bacilles species, a Mycobacterium species, a Pseudomonas, or a Streptococcus species.

26. The method of claim 25, wherein the Staphylococcus species is S. aureus or S. epidermidis.

27. (canceled)

28. The method of claim 24, wherein the candidate substance is a peptide, a peptidomimetic, or an organopharmaceutical.

29. The method of claim 24, wherein MgrA polypeptide or fragment binding to DNA is measured by a gel mobility shift assay, a South-Western blot, fluorescence anisotropy or FRET assay.

30. The method of claim 24, wherein at least steps (a) and (b) are performed in a cell-free system.

31. The method of claim 24, wherein at least steps (a) and (b) are performed in a bacterial cell.

32. The method of claim 24, wherein said MgrA polypeptide or fragment contains an oxidized Cys12 residue, a reduced Cys12 residue, or an alkylated Cys residue.

33. The method of claim 24, further comprising contacting said MgrA polypeptide or fragment with an oxidizing agent or an alkylating agent.

34. The method of claim 33, wherein said oxidizing agent or alkylating agent is added (i) prior to step (b) or (ii) after step (b) and before step (c).

35. The method of claim 33, wherein said oxidizing agent is hydrogen peroxide, an organic hydroperoxide, nitric oxide or superoxide.

36. The method of claim 33, wherein said alkylating agent is selected from the group consisting of an aldehyde, a halomethyl ketone, a diazomethyl ketone, an acyloxymethyl ketone, an epoxide and a vinyl sulfone.

37. A method of identifying a modulator of bacterial MgrA function comprising:
(a) providing an MgrA polypeptide or fragment thereof that comprises Cys12;
(b) contacting said MgrA polypeptide or fragment with a candidate substance; and
(c) assessing the oxidation state or alkylation state of a cysteine residue corresponding to that found at Cys12 of Staphylococcus aureus MgrA in said MgrA polypeptide or fragment,
wherein a change in the oxidation or alkylation state of Cys12 of said MgrA polypeptide or fragment, as compared to the alkylation or oxidation state of Cys12 of said MgrA polypeptide or fragment in the absence of said candidate substance, identifies said candidate substance as a modulator of bacterial MgrA function.

38. The method of claim 37, wherein the MgrA is from a Staphylococcus species, a Bacilles species, a Mycobacterium species, a Pseudomonas, or a Streptococcus species.

39. The method of claim 38, wherein the Staphylococcus species is S. aureus or S. epidermidis.

40. (canceled)

41. The method of claim 37, wherein the candidate substance is a peptide, a peptidomimetic or an organopharmaceutical.

42. The method of claim 37, wherein assessing the oxidation state of Cys12 comprises NBD assay or DTNB assay.

43. The method of claim 37, wherein at least steps (a) and (b) are performed in a cell-free system.

44. The method of claim 37, wherein at least steps (a) and (b) are performed in a bacterial cell.

45. The method of claim 37, wherein said MgrA polypeptide or fragment contains an oxidized Cys12 residue, a reduced Cys12 residue or an alkylated Cys12 residue.

46. The method of claim 37, further comprising contacting said MgrA polypeptide or fragment with an oxidizing agent or an alkylating agent.

47. The method of claim 46, wherein said oxidizing agent or alklylating agent is added (i) prior to step (b) or (ii) after step (b) and before step (c).

48. The method of claim 46, wherein said oxidizing agent is hydrogen peroxide, an organic hydroperoxide, nitric oxide or superoxide.

49. The method of claim 46, wherein said alkylating agent is selected from the group consisting of an aldehyde, a halomethyl ketone, a diazomethyl ketone, an acyloxymethyl ketone, an epoxide and a vinyl sulfone.

50-69. (canceled)

70. A method of reducing the virulence of a bacterium comprising contacting said bacterium with an agent that inhibits the expression of MgrA.

71. The method of claim 70, wherein the bacterium is a Staphylococcus species, a Bacilles species, a Mycobacterium species, a Streptococcus species or a Pseudomonas species.

72. The method of claim 70, wherein the inhibitor is an organopharmaceutical, protein, peptide, peptidomimetic or nucleic acid.

73. The method of claim 70, wherein the bacterium is in an animal host.

74. The method of claim 73, wherein the animal host is a human or a cow.

75. The method of claim 70, wherein the bacterium is a multi-drug resistant strain.

76. A method of reducing the virulence of a bacterium comprising contacting the bacterium with an agent that decreases MgrA binding to DNA or promotes MgrA dissociation from DNA.

77. The method of claim 76, wherein the bacterium is a Staphylococcus species, a Bacilles species, a Mycobacterium species, a Streptococcus species or a Pseudomonas species.

78. The method of claim 76, wherein the inhibitor is an organopharmaceutical, protein, peptide, peptidomimetic or nucleic acid.

79. The method of claim 76, wherein the bacterium is in an animal host.

80. The method of claim 79, wherein the animal host is a human or a cow.

81. The method of claim 76, wherein the bacterium is a multi-drug resistant strain.

82. A method of reducing the virulence of a bacterium comprising contacting said bacterium with an agent that promotes oxidation or akylation of a cysteine residue corresponding to that found at Cys12 of Staphylococcus aureus MgrA or inhibits the reduction of an oxidized cysteine residue corresponding to that found at Cys12 of Staphylococcus aureus MgrA.

83. The method of claim 82, wherein the bacterium is a Staphylococcus species, a Bacilles species, a Mycobacterium species, a Streptococcus species or a Pseudomonas species.

84. The method of claim 82, wherein the inhibitor is an organopharmaceutical, protein, peptide, peptidomimetic or nucleic acid.

85. The method of claim 82, wherein the bacterium is in an animal host.

86. The method of claim 85, wherein the animal host is a human or a cow.

87. The method of claim 82, wherein the bacterium is a multi-drug resistant strain.

88. A method of reducing the virulence of a bacterium comprising contacting said bacterium with an agent that activates the expression and/or function of MgrA.

89. A method of reducing the virulence of a bacterium comprising contacting said bacterium with an agent that activates the expression and/or function of a reactive-oxygen sensing protein in the bacterium.

90. A method of reducing the virulence of a bacterium comprising contacting said bacterium with an agent that modulates the expression and/or virulence-regulating function of a reactive-oxygen sensing protein in the bacterium.
